(19) United States
(12) Patent Application Publication
(10) Pub. No.: US 2021/0039089 A1
(43) Pub. Date: Feb. 11, 2021

(54) DEVICES, KITS, AND METHODS FOR LABEL-FREE SEPARATION AND SUBTYPING OF RARE CELLS

(71) Applicant: University of Georgia Research Foundation, Inc., Athens, GA (US)

(72) Inventors: Leidong Mao, Watkinsville, GA (US); Yang Liu, Athens, GA (US)

(21) Appl. No.: 16/986,930

(22) Filed: Aug. 6, 2020

Related U.S. Application Data
(60) Provisional application No. 62/883,614, filed on Aug. 6, 2019.

(51) Int. Cl. B01L 3/00; C12M 3/06

(52) U.S. Cl. CPC B01L 3/50273; B01L 3/502715; B01L 3/502738; C12M 23/16; B01L 2200/142; B01L 2200/16; B01L 2400/084; B01L 2300/0816; B01L 2300/0864; B01L 2300/0887; B01L 2300/087; B01L 2300/161; B01L 2400/0688; B01L 2300/0645

(57) ABSTRACT

The present disclosure provides devices, kits, and methods for enriching/separating and/or subtyping target cells from a biological sample, such as circulating tumor cells or other types of rare cells or differentiating cells. Devices, kits, and methods of the present disclosure utilize a created chemogradient to modulate movement of target and/or non-target cells in a sample based on attraction and/or repulsion to certain chemical compounds to separate and subtype cells in a sample.

### CROSS-REFERENCE RELATED APPLICATION

This Application claims the benefit of U.S. Provisional Application entitled “DEVICES, KITS, AND METHODS FOR LABEL-FREE SEPARATION AND SUBTYPING OF CIRCULATING TUMOR CELLS FROM BIOLOGICAL SAMPLES,” having Ser. No. 62/883,614, filed on Aug. 6, 2019, which is incorporated herein by reference in its entirety.

### STATEMENT REGARDING FEDERALLY SPONSORED RESEARCH OR DEVELOPMENT

This invention was made with Government support under Grant Nos. 1150042, 1648035, and 1659525, awarded by the National Science Foundation and under Grant Nos. GM104528 and TR002378, awarded by the National Institutes of Health. The Government has certain rights in the invention.

### BACKGROUND

Various types of rare circulating cells found in a sample can provide potentially important information about a patient and/or status of cells in the sample. There is a continual search for technologies for sensitive and effective approaches for enrichment, separation, isolation, and characterization of various types of target cells from a sample. Some such target cells could include, but are not limited to stem cells, differentiating cells, disease cells, circulating tumor cells (CTCs), etc. CTCs are cancer cells that are detached from primary solid tumors and carried through the vasculature to potentially seed distant site metastases in vital organs, representing the main cause of death in cancer patients. Molecular assessments of CTCs not only could benefit basic cancer research, but also might eventually lead to a more effective cancer treatment.

However, one major limitation to the study of CTCs has been the limited availability of viable CTCs for investigations, due in part to the small patient blood volumes that are allowable for research, which usually yielded less than 100 CTCs from 1 mL of whole blood. As a result, technologies are needed that facilitate separation of these rare cells from blood, and important performance criteria for these technologies include the ability to process a significant amount of blood quickly, a high recovery rate of CTCs, a reasonable purity of isolated cancer cells, and cell integrity for further characterization.

Label-based CTC separation technologies were developed to selectively enrich a subset of CTCs from blood, primarily through the use of specific biological markers including epithelial cell adhesion molecule (EpCAM). These antigen-based labels were a rate-limiting factor in effective CTC separation, as the inherent heterogeneity of CTCs render these technologies ineffective for general use. The vast array of various biomarkers that might or might not be expressed, and which cannot be predicted to remain expressed in CTCs undergoing Epithelial-to-Mesenchymal Transitions (EMT) are cumbersome and confounding in these label-based methods. Furthermore, most label-based technologies do not conveniently enable comprehensive molecular analysis of separated CTCs because they are either dead or immobilized to a surface. Thus, a variety of label-free methods have been developed to exploit specific physical markers in order to deplete non-CTCs in blood and thereby enrich cancer cells. While such methods may be used to separate CTCs based upon, for example, size, the existence of large white blood cells, such as monocytes, that may have overlapping sizes with CTCs complicate these label-free methods and reduce the purity of the sample obtained. Other devices have attempted to incorporate two or more of these methods, but still suffer from the time-consuming and laborious sample preparation due to the complications discussed above for labeling CTCs. Furthermore, previous methods do not allow further separation and subtyping of circulating CTCs. Studies have shown that the cells in primary tumors are heterogeneous and that CTC's from such tumors have similar heterogeneity, with some CTC's having a highly invasive/metastatic potential and other CTS's displaying a less invasive/more benign phenotype. Additional knowledge about the type and percentage of a patients CTC's could aid in cancer treatment strategy and identification of high-risk patients.

There remains a need for improved devices and methods for separating circulating tumor cells that overcome these shortcomings as well as methods for subtyping CTC's within a biological sample.

### SUMMARY

In accordance with the purpose(s) of the disclosure, as embodied and broadly described herein, the disclosure, in various aspects, relates to microfluidic devices, kits including such devices, and methods of using microfluidic devices that overcome one or more of the above deficiencies. The devices and methods can combine filters, chemo-modulatory separation, and to provide for enrichment, separation, and classification of rare cells.

Embodiments of devices of the present disclosure for enrichment, separation, and subtyping of circulating target cells in a biological sample fluid include a microfluidic chip substrate having a plurality of microfluidic channels formed thereon. According to embodiments, the microfluidic channels include: a main channel having a first end and a second end, a first inlet at the first end wherein the inlet is configured to flow a first fluid into the main channel, and a first outlet at the second end configured to collect contents of the first fluid that exit the main channel; a first outer channel oriented substantially parallel to and on a first side of the main channel and having a first end and a second end, a second inlet at the first end of the first outer channel wherein the second inlet is configured to flow a second fluid into the first outer channel, and a second outlet at the second end of the first outer channel; and a first-side plurality of microchannels connecting the main channel to the first outer channel and oriented substantially perpendicular to the main channel and first outer channel, wherein the first-side plurality of microchannels are in fluidic communication with both the main channel and the first outer channel. In embodiments, devices of the present disclosure also include one or more first-side collection channels located in between and oriented substantially parallel to both the main channel and first outer channel and in fluidic communication with the first-side plurality of microchannels, the one or more first-side collection channels each having a first and second end and each first-side collection channel being in fluidic communication with a first flushing port at the first end and each having an individual collection outlet at the second end.

In embodiments, the devices of the present disclosure can also include a second outer channel on a second side of the main channel opposite the first outer channel, the second outer channel oriented substantially parallel to the main channel and first outer channel and having a first end and a second end, a third inlet at the first end of the second outer channel wherein the third inlet is configured to flow a third fluid into the second outer channel, and a third outlet at the second end of the second outer channel and a second-side plurality of microchannels connecting the main channel to the second outer channel and oriented substantially perpendicular to the main channel and second outer channel, wherein the microchannels are in fluidic communication with the main channel and the second outer channel. Devices of the present disclosure can also include one or more second-side collection channels located in between and oriented substantially parallel to both the main channel and second outer channel and in fluidic communication with the second-side plurality of microchannels, the one or more second-side collection channels each having a first and second end and each second-side collection channel being in fluidic communication with a second flushing port at the first end and each having an individual collection outlet at the second end.

The present disclosure also provides kits for enrichment, separation, and subtyping of circulating target cells in a biological sample fluid where the kits include a device according to the present disclosure, at least one chemo-modulatory fluid or instructions for preparing at least one chemo-modulatory fluid, and instructions for use of the device and the at least one chemo-modulatory fluid to separate target cells from a biological sample fluid. In embodiments where the device has both first and second outer channels, kits of the present disclosure can also include a second chemo-modulatory fluid or instructions for preparing a second chemo-modulatory fluid, and instructions for use of the device and the chemo-modulatory fluids.

Methods of the present disclosure for separating target cells in a biological sample, according to some embodiments, include introducing a chemo-modulatory fluid in the first inlet of the main channel or the second inlet of the first outer channel of a device of the present disclosure, such that the chemo-modulatory fluid flows in the device and establishes a chemogradient between the main channel and the first outer channel of the device and introducing and flowing a biological sample fluid into the other of the main channel or first side channel that is not flowing the chemo-modulatory fluid. Methods further include allowing and detecting migration of target cells and non-target cells in the biological sample fluid in response to the chemogradient, such that target cells in the sample move toward or away from the chemo-modulatory fluid in response to the chemogradient, non-target cells in the sample migrate toward or away from the chemo-modulatory fluid in response to the chemogradient, or both, and wherein the movement of the target cells is distinguishable from the movement of the non-target cells based on one or more of speed and distance of migration of the cells across the chemogradient in the device via the plurality of microchannels. Embodiments of methods of the present disclosure also include introducing and flowing a flushing fluid from the first flushing port through the one or more first-side collection channels such that any target or non-target cells located in the one or more first-side collection channels between the main channel and the first outer channel are flushed by the flushing fluid to the individual collection outlets and are separated into different collection outlets based on distance of migration within the device.

According to some embodiments of the present disclosure where a device of the present disclosure is used that includes a second outer channel, a second-side plurality of microchannels, and second-side collection channels, methods can also include introducing a chemo-modulatory fluid in either the first inlet of the main channel or in both of the second and third inlets of the first and second outer channels, respectively, such that the chemo-modulatory fluid flows in the device and establishes a chemogradient between the main channel and the first and second outer channels of the device, and introducing and flowing a biological sample fluid comprising target cells into the other of the main or first and second outer channel that is not flowing the chemo-modulatory fluid. Such methods also include allowing and detecting migration of target cells and non-target cells in the biological sample fluid in response to the chemogradient, such that target cells in the sample move toward or away from the chemo-modulatory fluid in response to the chemogradient, non-target cells in the sample migrate toward or away from the chemo-modulatory fluid in response to the chemogradient, or both, and wherein the movement of the target cells is distinguishable from the movement of the non-target cells based on one or more of speed and distance of migration of the cells across the chemogradient in the device via the plurality of microchannels. These methods can also include introducing and flowing a flushing fluid from either or both the first and second flushing port through either or both of the one or more first-side collection channels and one or more second-side collection channels such that any target or non-target cells located in the one or more first-side and second-side collection channels between the main channel and the first and second outer channels are flushed by the flushing fluid to the individual collection outlets and are separated into different collection outlets based on distance of migration within the device.

Other systems, methods, devices, features, and advantages of the devices and methods will be or become apparent to one with skill in the art upon examination of the following drawings and detailed description. It is intended that all such additional systems, methods, devices, features, and advantages be included within this description, be within the scope of the present disclosure, and be protected by the accompanying claims

### BRIEF DESCRIPTION OF THE DRAWINGS

Further aspects of the present disclosure will be more readily appreciated upon review of the detailed description of its various embodiments, described below, when taken in conjunction with the accompanying drawings. The components in the drawings are not necessarily to scale, emphasis instead being placed upon clearly illustrating the principles of the present disclosure. Moreover, in the drawings, like reference numerals designate corresponding parts throughout the several views.

FIGS. 1A-1D provide an overview of embodiments of the microfluidic device of the present disclosure and methods of use for enrichment, separation, and subtyping of circulating target cells in a sample. FIG. 1A is a schematic illustration of the cell migration process under chemo-attractant in a device of the present disclosure. FIG. 1B provides digital images (top panel) of the PDMS-based microfluidic device (top right, the colored ink indicating the channel geometry) and close-up images of the microfluidic structure of the devices (bottom panel). FIG. 1C illustrates top-view schematic drawings of the microchannel configuration of the microfluidic device of the present disclosure and two different modes of operation (top, with a sample fluid containing cells loaded in the outer/side channels and chemo-modulatory fluid loaded in main/middle channel; bottom, with sample fluid containing cells loaded in main/middle channel and chemo-modulatory fluid(s) loaded in the outer/side channels). FIG. 1D illustrates magnified images of the migration channels (plurality of microchannels) separated by collection channels. On the left are phase-contrast images of the migration pattern of MDA-MB-231 breast cancer cell line. The middle channel was loaded with 10% FBS cancer cell chemo-attractant. Right, experimental fluorescence images show the migration pattern of MDA-MB-231 using 10% FBS as chemo-attractant.

FIGS. 2A-2? Illustrate fluidic characterization of an embodiment of the microfluidic device of the present disclosure. FIG. 2A illustrates a schematic simulation of the flow profile in the microfluidic device and individual channels of the device with vectors representing flow direction. The close up of one side of the device illustrates directions in the flow channels and migratory microchannels, with the left window indicating the flow direction in the microchannels nearest the 0% FBS loading channel and the right-hand window showing the flow profile in the migration microchannels channel nearest the 10% FBS loading channel. FIG. 2B illustrates a simulation of chemo-attractant concentration gradient near the middle of the microfluidic channel. The diffusion ecoefficiency of FBS was set to 61 μm2/s. The viscosity of the medium was 0.94 cP. FIG. 2C are graphs of the FBS concentration gradient over time from 0-24 hours. The left-hand graph is a time-lapse plot of FBS concentration across the middle of black line in FIG. 2B. The concentration gradient across the windows was quickly stabilized within 4 minutes when the flow rate was 0.1 μL/min. The right-hand graph illustrates concentration distributions of FBS in the middle of #1, #2 and #3 observation window at t=24 hour. The concentration distributions of FBS were similar in the transverse direction.

FIGS. 3A-3F illustrate a migration assay of breast cancer cell line MDA-MB-231 with approximately 5000 cancer cells suspended in FBS-free medium loaded into the outer channel, while the middle channel is loaded 10% FBS medium. The mediums were refreshed with a continuous flow at v=0.1 μL/min. The left-hand image in FIG. 3A is microscopic image of immunofluorescence stained cancer cells after 24-hour's migration. Migratory cells deformed and squeezed into the plurality of microchannels in the migration channels (#2, #4, #6 and #8) and moved to the place with higher FBS concentration. Non-migratory cells stayed in the cell loading channel. The migration direction was from top to bottom. All the cells were fixed and stained with EpCAM, vimentin, and DAPI (right-hand close-up image). FIG. 3B illustrates final positions of cancer cells at different FBS concentrations (0%, 5%, and 10%) in the middle channel. The time of the migration assay ranged from 0 hours to 24 hours. Lines in the box graph indicate the mean values of the final position. The left-hand image in FIG. 3C is a scatter dot plot of the Vimentin fluorescence intensity of cancer cells (n=485, time=24 hours). The shade of the dots indicates EpCAM fluorescence intensity. Both vimentin and EpCAM intensity increased in the collection channel, whereas the intensities for both markers decreased in the migration channel (shaded rectangles). The left-hand image in FIG. 3C is a graph illustrating average fluorescent intensities of vimentin (upper line) and EpCAM (lower line) in each channel. Signal oscillations for vimentin and EpCAM could be observed across the channel, while the mean value of the vimentin was larger than of EpCAM. FIG. 3D is a scatter dot plot illustrating that cells in the migration channel had a relatively larger aspect ratio and lower solidity. The scatter dot plot of FIG. 3E represents the relationship between aspect ratio and vimentin intensity of cancer cells (n=485). R² of the linear regression fitting is 0.001. FIG. 3F is a series of scatter dot plots of cancer cells in different channels (collection channel, left, and migration channel, right) indicating the linear relationship between vimentin intensity and aspect ratio. The R² values for collection channel #1, 3, 5, and 7 are 0.94, 0.95, 0.96, and 0.95, respectively. The R² values for migration channel #2, 4, 6, and 8 are 0.97, 0.94, 0.95, and 0.99, respectively.

FIGS. 4A-4H illustrate migration assays of breast, lung and prostate cancer cell lines in the device of the present disclosure under optimized conditions (flow rate=0.1 μL/min, FBS concentration=10%, and t=24 hour). After 24 hours, cells were fixed and stained with four markers (EpCAM, vimentin, CD44, and DAPI). FIG. 4A, left, is a microscopic image of immunofluorescence stained MDA-MB-231 cell line, while right shows scatter dot plots of 8 different cancer cell lines (lung: A549, H3122, and H1299; breast: HCC70, HCC1806, MCF7, and MDA-MB-231; prostate: PC-3). The final positions in the Y direction were calculated using the nucleus signal (DAPI). Black lines indicate the average final position. FIG. 4B is a table showing the percentage of cancer cells in each channel after 24 hours. Channel #1 was the cell loading channel, Channel #2, #4, #6, and #8 were migration channels, Channel #1, #3, #5, and #7 were collection channels, and Channel #9 is the FBS loading channel. FIG. 4C are a series of immunofluorescence images of eight cancer cell lines after migration. 4 channels were used in the immunofluorescence staining, including Epithelial marker EpCAM (column 1), mesenchymal marker vimentin (column 3), cancer stem cell marker CD44 (column 2), and nucleus marker DAPI (column 4), with the overlay in column 5. The non-migratory cell lines (MCF7, H3122, and HCC70) were identified when the average final position is smaller than 300 μm, and the migratory cell lines (MDA-MB-231, PC-3, A549, H1299, and HCC1806) were represented when the average final position was larger than 300 μm. FIG. 4D is a web plot illustrating average intensities of 3 makers, EpCAM, Vimentin, and CD44 for 8 cancer cell lines (n=2000). The intensities were normalized using the intensity maxima (EpCAM: H3122, vimentin: MDA-MB-231, and CD44: H3122). FIGS. 4E-G are a series of web-plots illustrating average aspect ratio (4E), solidity (4F) and circularity (4G) of 8 cancer cell lines (n=2000). FIG. 4H is a series of scatter plots of 8 cancer cell lines to correlate the average Y positions to mean aspect ratio, CD44, vimentin and EpCAM fluorescence intensity. The lines indicate the linear regression fit with R²=0.82, 0.74, 0.41 and 0.55, respectively. And P=0.002, 0.006, 0.036 and 0.086, respectively. FIG. 4I illustrates average aspect ratio and vimentin intensity of high migratory cell lines (H1299, PC-3, HCC1806, and MDA-MB-231) in different channels. FIG. 4J are images of separation of migratory cell line (-MB-231) from non-migratory cell line (MCF7) using MChip under optimized conditions (flow rate=0.1 μL/min, FBS concentration=10%, and t=24 hour). The arrow indicates the migration direction. The white dash line represents the starting point of channel #2.

FIGS. 5A-5E illustrate cell viability and proliferation assay of H1299 cells. a-b short term viability of cancer cells in the microfluidic chip after 6 hours, 12 hours and 24 hours' migration under optimized conditions (flow rate=0.1 μL/min, FBS concentration=10%). a The cell viability of H1299 lung cancer cells at 6 hours, 12 hours and 24 hours' migration was determined to be 98.63%±0.47%, 98.33±0.21% and 96.67±0.63%, respectively. b Representative images of Live/Dead staining on MChip. c-e After 24 hours' migration, H1299 cells from channel #1, #3 and #5 were retrieved with Trypsin-EDTA for 8 minutes and cultured in the 48-well plate for 72 hours. c Representative images of retrieved H1299 cell culture over 72 hours. Live/Dead staining of the culture cells shows excellent cell viability. d Total number of cells quantified from cell culture imaging for cells collected from channel #1 (red), #3 (blue) and #5 (green) at 0 hour and the change in counts at 72 hours. e Growth rate of retrieved cells from different collection channels ((cell number at 72 hr-cell number at 0 hr)/cell number at 72 hr).

FIGS. 6A-6H illustrate purification of circulating tumor cells isolated from human blood using iFCS separation and an embodiment of a device of the present disclosure. FIG. 6A is images validation of combination effect of using iFCS and a device of the present disclosure with cultured cancer cells spiked into healthy blood. Left, an iFCS device was used to recover more than 99.2% spiked cancer cells and deplete many WBCs (˜500 cells per mL). Collected cancer cells and WBCs were injected into the “MChip” of the present disclosure to further purify CTC by removing most remaining WBCs based on different migratory abilities, as shown in the right image. Migratory cancer cells could be retrieved with high purity. FIG. 6B is a graph comparing migratory abilities for cells before and after iFCS processing. The two groups have similar median (black line) and mean value (star symbol), which indicates that iFCS has negligible effect on the migratory ability of cancer cells. FIG. 6C illustrates WBC (n=-5000, channel #1 loading) migration assays under control (FBS only), WBC chemo-attractant (FM LP) and WBC chemo-repellent (Slit2). FBS (10%) or Slit2 (5 μg/mL) were loaded into the middle channel (channel #9), while FMLP (200 ng/mL) were filled in the cell loading channel (channel #1). Only the WBCs of >200 μm Y position were counted. Left, the graph shows positions of individual WBCs and the rectangular symbols indicate the mean value. Right, images of WBCs 24-hour migration assay using Slit2 (5 μg/mL) in 10% FBS. Flow rate v=0.1 μL/min. WBCs were stained with cell tracker red. Fluorescence images were taken at 0 hour and 24 hours. Circles indicate the migratory WBCs. FIG. 6D is a graph illustrating breast cancer cells, MDA-MB-231, loaded into channel #1 with Slit2 (5 μg/mL) in 10% FBS in the middle channel. Flow rate v=0.1 μL/min. The graph indicates positions of MDA-MB-231 and the rectangular symbols indicate the mean value. FIG. 6E is an image of WBCs (red) and MDA-MB-231 (green) mixture (1:1) migration assay with middle channel (channel #9) filled with 10% FBS and Slit2 (5 μg/mL). At 24 hours, most of the migratory cancer cells moved into the migration/collection channels with ˜2 WBCs contamination. FIG. 6F illustrates a WBC migration assay using FBS, FMLP, and Slit2. WBCs (n=˜2500) loaded into the top edge of the middle channel. 10% FBS or Slit2 were loaded into top channel #1, while FMLP was filled into bottom channel #1. The rectangular symbols represent the mean value of final positions. FMLP had a significant performance in inducing cells toward the bottom channel. FIG. 6G are images illustrating cancer cell separation according to methods of the present disclosure under effect of chemo modulatory compounds, FBS (10%, top channel #1) and FMLP (200 ng/mL, bottom channel #1). MDA-MB-231 cells and WBCs were mixed with a 1:1 ratio and then loaded into the top edge of the middle channel (bottom arrow the bright-field image). The fluorescence image shows the final positions of the cell mixture (t=24 hours). Immunofluorescence staining was used to identify MDA-MB-231 (Vimentin, green, mostly upper portion of device) and WBCs (CD45, red, mostly lower). The arrows indicate the migration directions. FIG. 6H is a graph illustrating cell distribution after 24-hour migration showing that most of the MDA-MB-231 cells (light gray) moved to the top side while most of the WBCs (dark gray) move to the bottom side. Only about 0.4% of WBCs migrated to the top channel.

FIG. 7 is a graph illustrating cell migratory behaviors relative to glass coating methods (of the columns) of collagen vs. poly-L-Lysine.

FIG. 8 is a scatter dot plot (left) and a graph (right) illustrating that cells in the migration channels had a relatively higher “Vimentin to EpCAM” ratio as compared with the cells in the collection channels and showing ratios were larger for cells with longer migration distance (Channel #6-9).

FIG. 9 are graphs illustrating position of cells in migration channels as a function of aspect ratio (left) and solidity (right), showing that migratory cells typically have higher aspect ratio and lower solidity.

FIG. 10 is a series of dot plots illustrating the relationship of solidity to vimentin intensity for cells in migration channels.

FIG. 11 illustrates EpCAM intensity (left) and vimentin intensity (right) of different cancer cell lines tested in the devices of the present disclosure.

FIG. 12 are graphs illustrating correlation between average final position and circularity (aspect ratio) (left) and solidity (right).

FIG. 13 is a series of scatter dot plots illustrating vimentin intensity relative to final position for 4 different cell types.

FIG. 14 is a graph illustrating the lack of negative effect of FMLP (neutrophil chemo-attractant) vs control on migration of target cancer cells.

### DETAILED DESCRIPTION

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, material science, biology, molecular biology, microfluidics, and the like, which are within the skill of the art. Such techniques are explained fully in the literature.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20-25° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only and is not intended to be limiting. It is also possible in the present disclosure that steps can be executed in different sequence where this is logically possible.

All publications and patents cited in this specification are cited to disclose and describe the methods and/or materials in connection with which the publications are cited. Publications and patents that are incorporated by reference, where noted, are incorporated by reference as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. Any terms not specifically defined within the instant application, including terms of art, are interpreted as would be understood by one of ordinary skill in the relevant art; thus, is not intended for any such terms to be defined by a lexicographical definition in any cited art, whether or not incorporated by reference herein, including but not limited to, published patents and patent applications. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

It should be noted that ratios, concentrations, amounts, and other numerical data can be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to about 5%” should be interpreted to include not only the explicitly recited values of about 0.1% to about 5%, but also include individual values (e.g., 1%, 2%, 3%, and 4%) and the sub-ranges (e.g., 0.5%, 1.1%, 2.2%, 3.3%, and 4.4%) within the indicated range. Where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In some embodiments, the term “about” can include traditional rounding according to significant figures of the numerical value. In addition, the phrase “about ‘x’ to ‘y’”, where ‘x’ and ‘y’ are numerical values, includes “about ‘x’ to about ‘y’”.

Prior to describing the various embodiments, the following definitions are provided and should be used unless otherwise indicated.

### Definitions

Unless defined otherwise, all technical and scientific terms used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

The articles “a” and “an,” as used herein, mean one or more when applied to any feature in embodiments of the present invention described in the specification and claims. The use of “a” and “an” does not limit the meaning to a single feature unless such a limit is specifically stated. The article “the” preceding singular or plural nouns or noun phrases denotes a particular specified feature or particular specified features and may have a singular or plural connotation depending upon the context in which it is used.

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of”.

In this disclosure, “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially of” or “consists essentially” or the like, when applied to methods and compositions encompassed by the present disclosure have the meaning ascribed in U.S. Patent law and the term is open-ended, allowing for the presence of more than that which is recited so long as basic or novel characteristics of that which is recited is not changed by the presence of more than that which is recited, but excludes prior art embodiments.

As used herein, “about,” “approximately,” “substantially,” and the like, when used in connection with a numerical variable, can generally refers to the value of the variable and to all values of the variable that are within the experimental error (e.g., within the 95% confidence interval for the mean) or within +1-10% of the indicated value, whichever is greater. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “optional” or “optionally” indicates that the subsequently described event or circumstance can or cannot occur, and that the description includes instances where said event or circumstance occurs and instances where it does not.

As used herein, “kit” refers to a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” refers to documents describing relevant materials or methodologies pertaining to a kit. These materials may include any combination of the following: background information, list of components and their availability information (purchase information, etc.), brief or detailed protocols for using the kit, troubleshooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents and are meant to include future updates.

As used herein, the term “biocompatible,” with respect to a substance or fluid described herein, indicates that the substance or fluid does not adversely affect the short-term viability or long-term proliferation of a target biological particle within a particular time range.

As used herein the term “chemogradient” refers to a fluid chemical gradient created in a device of the present disclosure characterized by a varying % concentration of a chemical compound (e.g., a chemo-modulatory compound) in one area of the device of the present disclosure to another. For instance, near where a chemo-modulatory fluid is introduced to the device the concentration of the chemo-modulatory fluid would be higher than in an area of the device further away from where the chemo-modulatory fluid was introduced.

The term “chemo-modulatory” as used herein refers to a chemical compound (e.g. a chemical compound in a fluid) that has the ability to modulate the movement of a cell within a fluid, e.g., either toward (attraction) or away from (repellant) the chemical compound. For instance, a chemo-attractant has the effect of attracting a certain cell or cell-type, while a chemo-repellent has the effect of repelling a certain cell or cell-type. In embodiments, a chemo-modulatory fluid can include one or more fluids, e.g., a compound that may be a chemo-attractant for a first type of cell and a different compound that may be a chemo-repellent for a different type of cell.

As used herein, channels that are “substantially parallel” refers to channels of a device of the present disclosure that, with respect to a reference channel, while not absolutely parallel throughout the entire device, are parallel for a majority of the length of the channel, particularly in a portion of the channel in which cell migration is being observed.

### Discussion

General Approach

In accordance with the purpose(s) of the present disclosure, as embodied and broadly described herein, embodiments of the present disclosure, in some aspects, relate to devices, kits and methods for enriching/separating target cells from a biological sample, such as but not limited to, a sample from a patient (e.g., a fluid sample from urine, blood, respiratory secretions, extracellular fluid, exudate, and the like). In embodiments, the target cells are circulating tumor cells (CTCs). Embodiments of such devices, kits, and methods can also further separate/differentiate target cells into subtypes for further classification, research, analysis, diagnostic purposes, and the like.

Devices, kits, and methods of the present disclosure utilize chemo-modulatory manipulation to sort and separate target cells, such as, but not limited to CTCs, from other cells/particles (e.g., white blood cells and other cells) in a biological sample based on differences in migratory phenotypes in response to certain chemicals (e.g., media, chemo-attractants, chemo-repellents, etc.). Embodiments of such devices, kits and methods facilitate separation of target cells from non-target cells in a biological sample fluid, and optionally further sub-classification of the target cells. In example embodiments, devices, kits, and methods of the present disclosure provide for separation of CTC's from white blood cells in biological samples and further subtyping of the CTC's (e.g., based on affinity for or repulsion by a particularly chemo-modulatory compound). Embodiments of the present disclosure are also advantageous in that neither short-term cell viability nor long-term proliferation of target cells are impacted. While the description and examples below generally illustrate and discuss the use of the device of the present disclosure where the target cells are CTCs, it will be understood and appreciated by a skilled artisan that the devices, kits, and methods of the present disclosure can be used to enrich, separate, and classify other types of target cells from samples. The devices and methods of the present disclosure are particularly useful for separation of rare circulating cell types with modulatory behaviors, such as differentiating cells (e.g., stem cells or other semi-potent or pluri-potent cells), cancer cells, disease cells, and the like.

Cancer is among the largest killers in the world. The American Cancer Society estimates there will be 1,762,450 new cases and 606,880 deaths in 2019 alone due to cancer. Of these deaths, ninety percent are predicted to be caused by metastatic cancers; any cancer that has spread from their primary tumor. If these metastatic cells can be separated and studied, great breakthroughs can be made in the fight against cancer. For cancer cells to metastasize they must be able to migrate and invade through a process called the invasion metastasis cascade; a process by which a cancer cell creates a secondary tumor. Primary tumors are heterogeneous and made of cancer stem cells (CSCs) and non-cancer stem cells (Non-CSCs), collectively circulating tumor cells (CTCs). CSCs intrinsically have a phenotype that makes them self-renewable, tumor initiating, motile, invasive and more resistant to apoptosis. The non-CSCs, on the other hand, are not initially invasive, and must have these needed traits induced through a process called epithelial-mesenchymal-transition (EMT). Thus, analysis and subtyping of types of CTCs obtained from a liquid biopsy can help identify the presence of and quantification of CTCs that are the most likely creators of secondary tumors.

A standard biopsy is a tissue sample taken directly from the tumor. This biopsy is a time-intensive procedure, with a high pain and risk factor, is invasive and provides only a localized understanding of the tumor. It does not provide a full picture of the cancer when it has metastasized. However, the liquid biopsy is a quick, easily obtainable, minimal risk and pain, minimally invasive and provides a comprehensive tissue profile of the cancer. The difficulty with liquid biopsies is separating the tumor cells from the rest of the blood and understanding what those cancer cells can tell us about the cancer prognosis.

Since the makeup of CTCs in a biological sample is quite heterogeneous (similar to cells in a primary tumor), with some having a very invasive phenotype and others being benign tumor cells, being able to separate the various types and further study them can help direct treatment decisions for each patient. For instance, this could provide the ability to determine which patients are high-risk vs. low risk based on the type and/or percentage of different types of CTCs in their bloodstream. Exposure of patients at low risk of developing metastasis to aggressive treatments, such as radiotherapy, may compromise the patients' ability to tolerate further treatment that may be necessary to combat de novo cancer in the future. Thus, this can help identify which patients are at risk of developing metastatic disease to provide them with effective treatment while also minimizing the overtreatment of patients who are not at risk with potentially harmful and costly therapies.

Previous methods to evaluate cancer cell migration have included various techniques, including scratch assays, cell-exclusion zone assays, microfluidic based assays, and Boyden Chamber assays. The most widely accepted cell migration technique is the Boyden Chamber assay. The classic Boyden Chamber system uses a hollow plastic chamber, sealed at one end with a porous membrane. This chamber is suspended over a larger well which may contain medium and/or chemo-attractants. Cells are placed inside the Chamber and allowed to migrate through the pores, to the other side of the membrane. However, prolonged studies for Boyden chamber are inefficient, due to the fact that the test-agent concentration will quickly equalize between the compartment below the membrane and the compartment above the membrane. Another disadvantage is the relative difficulty in setting up the transwells. It is also difficult to track the cell migration in real time.

Thus, in order to study the migration behavior of circulating tumor cells as well as separate the CTCs from a sample for further analysis, the devices, kits and methods of the present disclosure include a cell migration chip for enrichment, separation and subtyping of circulating target cells in a biological sample fluid as described herein. The devices, kits, and methods of the present example allow the separation/purification of target cells (e.g., circulating tumor cells) from a subject's (e.g., a cancer patient's) sample (e.g., blood sample) by utilizing the differential migratory preferences of target cells and non-target cells (e.g., cancer cells and white blood cells). These methods and devices/kits further provide the ability to subtype target cells (e.g., CTCs) based on their migratory phenotypes.

Microfluidic cell migration devices were developed and optimized as described in the examples below, and studies were conducted using embodiments of microfluidic devices of the present disclosure that allow for the migration of cells in a sample in response to a chemogradient created in the device by the introduction of chemo-modulatory fluids/compounds. Experiments included phenotypic characterization, interaction with white blood cells and studying the effect of chemo-migratory cell separation. Preliminary results demonstrated that the invasive phenotype compared to non-invasive phenotypes of cancer cells includes features such as, but not limited to, a larger area, higher speed and velocity, a similar solidity value, lower persistence, and a higher aspect ratio. Beginning studies show that blood cells and the chemo-migratory separation treatment have negligible effects on the motility of cancer cells meaning that this technology will help to increase the effectiveness of liquid biopsies used for identifying metastatic cancers, help to create specially targeted therapies for invasive cancers, as well as help with separation and typing of other race circulating target cells.

Chemo-Modulatory Compositions

The devices of the present disclosure described in greater detail below, apply the migratory behavior of cells in response to chemo-modulatory compounds—compounds that have an attractive/repellant effect on cells. In order to differentiate different types of cells in a sample, chemo-modulatory compounds with differential effects on the different types of cells can be employed. Embodiments of devices, kits, and methods of the present disclosure employ a chemo-modulatory fluid composition (e.g., a fluid including a chemo-modulatory compound and referred to herein as “chemo-modulatory fluid,” “chemo-modulatory composition,” etc.) and establishment of a chemogradient to separate target cells and non-target cells in a biological sample (e.g., blood sample) based on differences in migratory behavior in response to the chemogradient. The chemo-modulatory fluid composition can be one that attracts or repels the target and/or non-target cells. In embodiments, the chemo-modulatory fluid composition has differing (e.g., opposite) effects on target cells and non-target cells, e.g., it may attract target cells and repel non-target cells or vice versa. Also, in embodiments, two different chemo-modulatory fluids can be used in different areas of the device to create a bi-directional chemogradient. For instance, a first chemo-modulatory fluid may include a chemo-attractant for target cells (and an optional chemo-repellent for non-target cells) and a second chemo-modulatory fluid may include a chemo-attractant for non-target cells and an optional chemo-repellent for target cells, and other variations.

In embodiments of the present disclosure, the target cells are CTC's and the non-target cells are WBCs. In such embodiments, the chemo-modulatory fluid composition can include a CTC attracting compound and/or a WBC repelling compound. In embodiments, a first chemo-modulatory fluid can include a CTC attracting compound and an optional WBC repelling compound and a second chemo-modulatory fluid can include a WBC attracting compound and optionally a CTC repelling compound. Examples of various such compounds are provided in Table 1 below, with possible concentration ranges provided for some candidate compounds. While these are meant to be representative examples, those of skill in the art can provide different chemo-attracting and chemo-repelling compositions depending on the identity of the WBCs, CTCs, or other target and non-target cells to be separated/analyzed in the devices of the present disclosure.

TABLE 1

| | |
|---|---|
| Type | Concentration |

Chemo-attractant for cancer cells

| | |
|---|---|
| FBS (Fetal Bovine Serum) | 0%-20% |
| EGF (Epidermal Growth Factor) | 0-1000 ng/ml |
| CXCL12 (Recombinant Human SDF-1α) | 0-1000 ng/ml |
| CCL19 (chemokine ligand 19) | — |
| CCL21 (chemokine ligand 21) | — |
| CCL22 (chemokine ligand 22) | — |
| CCL25 (chemokine ligand 25) | — |
| CX3CL1 (fractalkine) | — |
| CCR5 (chemokine receptor type 5) | — |
| FGF (Fibroblast growth factor) | — |
| PDGF (platelet-derived growth factor) | — |
| IGF1 (Insulin-like growth factor-1) | — |
| CSF1 (Colony stimulating factor 1) | — |
| VEGFA (Vascular endothelial growth factor A) | — |
| TFGp (Transforming growth factor beta) | — |

Chemo-repellent for WBCs

| | |
|---|---|
| Slit2 (Slit guidance ligand 2) | 0-2000 ng/mL |
| | (e.g.,1000 ng/mL) |
| SDF-1 (Stromal cell-derived factor-1) | |

Chemo-attractant for WBCs

| | |
|---|---|
| FMLP (fMet-Leu-Phe) (also may inhibit | e.g., 100 nM |
| cancer migration) | |
| LTB4 (Leukotriene B4) | — |
| C5a (Complement component C5) | — |
| IL-8 (Interleukin) | — |

Now having described the general functionality of the device, embodiments of devices and kits of the present disclosure for enrichment and separation of circulating tumor cells (CTCs), are described below, as well as methods of their use for the separation, enrichment, and subtyping of target cells in a sample.

Microfluidic Separation Devices and Kits

In embodiments, devices of the present disclosure for enrichment and separation of target cells in a biological sample fluid include include a microfluidic chip substrate having a plurality of microfluidic channels formed thereon. In embodiments, the chip substrate can be any suitable substrate material, such as, but not limited to, glass, silicones, polymers such as, but not limited to, PDMS (polydimethylsiloxane), PMMA (Polymethyl methacrylate), PC (Polycarbonate), PS (Polystyrene), COC (Cyclic Olefin Copolymer), COP (Cyclic Olefin Polymer), and the like.

In some embodiments devices of the present disclosure are fabricated using photolithography and soft lithography as known to those of skill in the art. In an embodiment, the photoresist is spread uniformly on a silicon wafer with the help of spin coater. Next, a photomask is situated right above photoresist. The transparent part, which represents the channel, will be exposed and cured by the UV light and formed into a mold. Then PDMS can be poured on the mold, the PDMS will cure after approximately 3 hours baking in oven. Then, the PDMS is peeled off from the silicon, which is modified and bonded on the glass slide. The process is efficient and cost effective.

In embodiments, the channels on the microfluidic chip substrate include at least a main channel, a first outer channel substantially parallel to the main channel, a first-side plurality of microchannels fluidly connecting the main channel to the first outer channel and oriented substantially perpendicular to the main channel and first outer channel, and, optionally, one or more firs-side collection channels located in between and oriented substantially parallel to the main channel and first outer channel and also in fluidic communication with the first-side plurality of microchannels. In embodiments, the device can also include a second outer channel on a second side of the main channel opposite the first outer channel, a second-side plurality of microchannels connecting the main channel to the second outer channel, and optionally, one or more second-side collection channels in between and substantially parallel to the main channel and second outer channel. In embodiments in which there is both a first outer channel and second outer channel, the device can be configured such that the first side and second side of the device a substantially symmetrical (e.g., mirror images) on either side of the main channel.

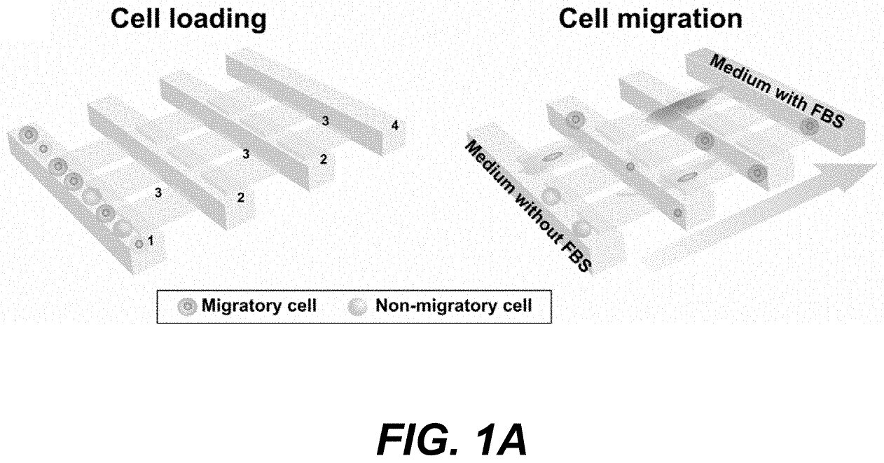

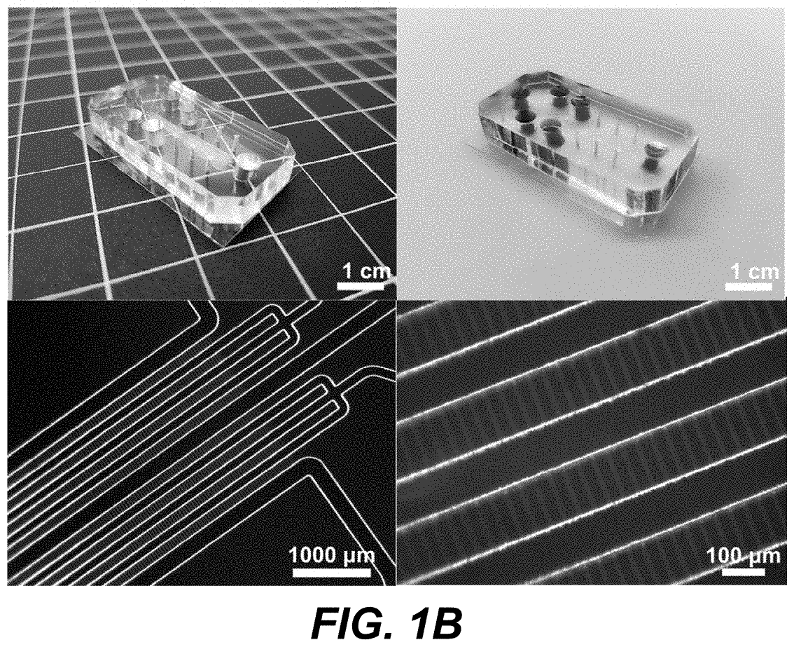

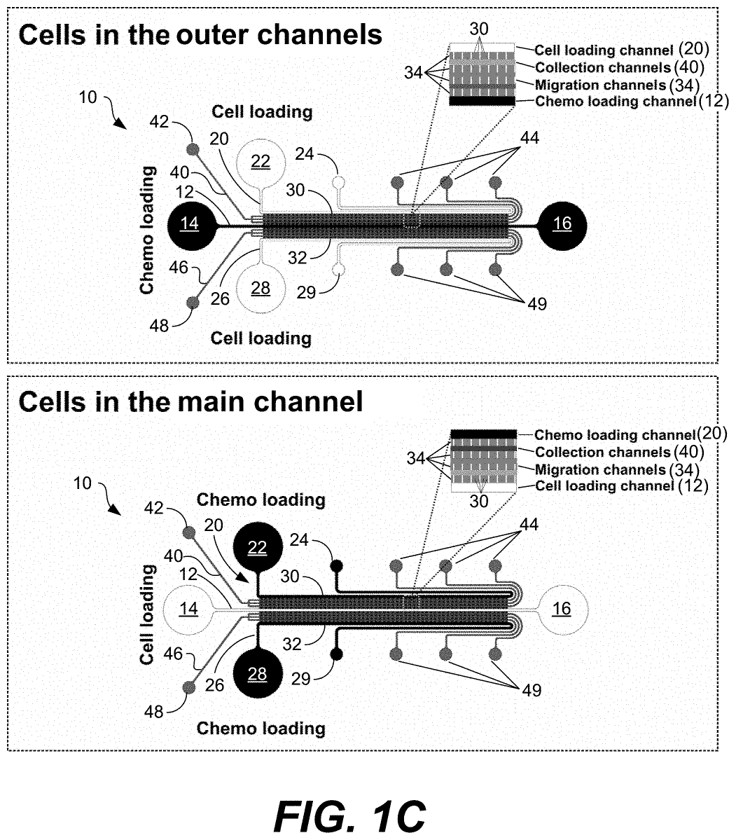

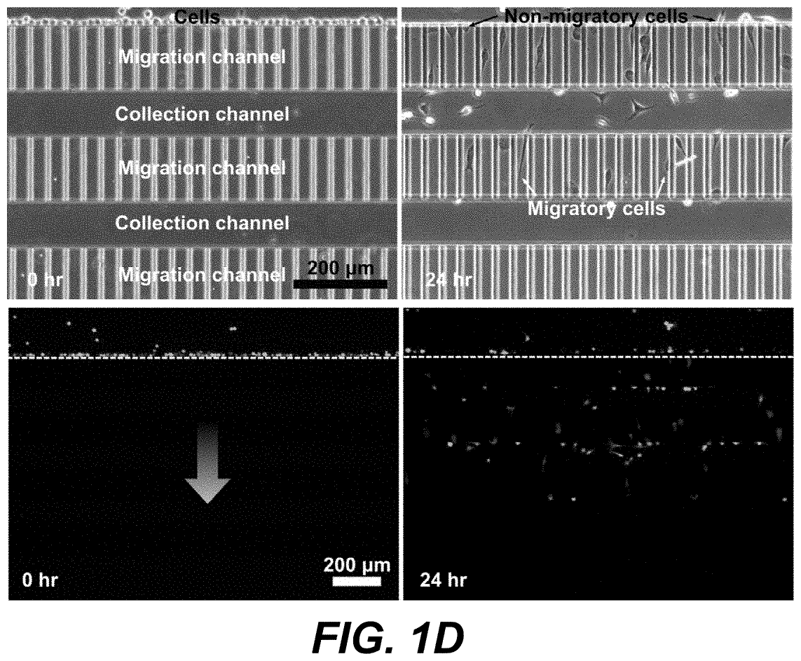

It should be understood that in various modes of use of the devices of the present disclosure either a chemo-modulatory fluid or a biological sample fluid can be introduced into/flowed through either of the main channel and/or outer channels of the device, thus in specific examples below, the present disclosure may sometimes refer to a channel of the device as a chemo-modulatory supply channel, or the like, and refer to another specific channel of the device as a cell flow channel or sample channel; however, it will be understood by a skilled artisan that the functionality of a channel in a specific embodiment does not necessarily define the identity of the channel, as a different fluid may be flowed through such channel at a different time or in a different mode of use. For purposes of illustrating the geometry of embodiments of devices of the present disclosure, the following description will refer to the schematic illustration in FIG. 1C of an embodiment of a device (and two possible modes of use), in which reference numerals are used to identify various parts.

As illustrated in FIG. 1C, a device 10 of the present disclosure the microfluidic channels on the microfluidic substrate include at least a main channel 12, a first outer channel 20, a first-side plurality of microchannels 30, and one or more first-side collection channels 40. The main channel 12 has a first end and a second end, with a first inlet 14 at the first end, the inlet configured to flow a first fluid into the main channel 12, and a first outlet 16 at the second end configured to collect contents of the first fluid that exit the main channel 12. The first outer channel 20 is oriented substantially parallel to and on a first side of the main channel 12 and has a first end and a second end. There is a second inlet 22 at the first end of the first outer channel, and the second inlet is configured to flow a second fluid into the first outer channel to a second outlet 24. The second outlet may be the second end of the first outer channel but may also curve around such that the outlet/port is doubled back near the inlet 22, as illustrated in FIG. 1C.

The device further includes a first-side plurality of microchannels 30 connecting the main channel 12 to the first outer channel 20 and oriented substantially perpendicular to the main channel and first outer channel. The microchannels are much smaller than the main and outer channels, with dimensions just sufficient to allow a single cell to pass through, where such cells may even have to deform slightly or squeeze to fit through. These plurality of microchannels 30 run perpendicularly between the main channel and outer channel, and any collection channels, fluidly connecting the other channels. The connected areas between the main, outer, and collection channels may be referred to herein as “migration areas” or “migration channels” (designated by reference number 34 in FIG. 1C) even though they refer to sections of the plurality of microchannels connecting the larger channels, and not to a channel itself. Depending on the number of collection channels, there can be a section of microchannels or “migratory section” (or “migration channel”) between each of the larger flow channels (as illustrated in the insets in FIG. 1C). However, it should be noted that the plurality of micro channels running perpendicular to the direction of the main, outer, and any collection channels are also fluidly connected with each other and the other channels, such that the channels of the device can all be fluidly connected. This arrangement where the first-side plurality of microchannels 30 are in fluidic communication with both the main channel 12 and the first outer channel 20, is such that when a chemo-modulatory fluid is flowed through either the main channel or the first outer channel, a fluid chemogradient is created between the main channel and the first outer channel, and such that when the biological sample fluid is flowed through the other of the main channel or first outer channel, target cells in the biological sample fluid migrate toward or away from the channel flowing the chemo-modulatory fluid in response to the chemogradient via the first-side plurality of microchannels, such that the target cells are separated from non-target cells in the biological sample fluid based on different migratory speeds and distances. The chemogradient will have a greater concentration of chemo-modulatory fluid closest to the channel in which it was introduced and is flowing and a lesser concentration in the microchannels and other channels further away from the channel in which it is flowing. Thus, when the biological sample fluid is flowed through the other of the main channel or first outer channel (that is not flowing the chemo-modulatory fluid), target cells in the biological sample fluid can migrate toward or away from the channel flowing the chemo-modulatory fluid in response to the chemogradient via the first-side plurality of microchannels. The movement of the target cells is distinguishable from movement (or lack thereof) of non-target cells. This migration allows the separation between target cells and non-target cells (as well as sub-separation of target cells) in the biological sample fluid based on different migratory speeds and distances between target vs non-target cells and even between different sub-types of target cells.

While the device can be operated as described above, with only a main channel and first outer channel connected by a plurality of microchannels, with collection of migrated cells via the first or second outlets from the main and/or outer channel, the device can also include one or more collection channels to assist collection of migrating cells at different migratory distances, times, and the like. Thus, in embodiments, such as illustrated in FIG. 1C, the device can also include one or more first-side collection channels 40 located in between and oriented substantially parallel to both the main channel and first outer channel and in fluidic communication with the first-side plurality of microchannels. The one or more first-side collection channels 40 each have a first and second end and each is in fluidic communication with a first flushing port 42 at the first end, where a fluid (e.g., a flushing fluid) can be introduced. Each collection channel can have an individual collection outlet 44 at the second end (which, like the outer channel outlet, may loop back around as illustrated for conservation of space on the chip). Each first-side collection channel is configured to flow a flushing fluid (e.g., a biocompatible fluid such as culture media, PBS, etc.) from the first flushing port 42 to the individual collection outlet(s) 44 such that any cells located in that first-side collection channel 40 are flushed by the flushing fluid to the individual collection outlet 44 for that first-side collection channel. Although in FIG. 1C only a single flushing port 42 is shown, that connects to 3 different collection channels, it will be understood that other embodiments can be contemplated where each collection channel has a separate flushing port. While the embodiment illustrated shows 3 collection channels per side (e.g., 6 collection channels total), there can be as few as zero or as many as about 6 collection channels on each side of the main channel of the device (e.g., 12 total; however, depending on the size of the chip substrate, a larger number of collection channels will result in the channels having decreased width).

As mentioned above, and as illustrated in FIG. 1C, devices of the present disclosure can include a second side of the device on the opposite side of the main channel, such that the second side of the device has a configuration that substantially mirrors the configuration of the first side of the device. Thus, in embodiments, devices of the present disclosure include a second outer channel 26 on a second side of the main channel 12 opposite the first outer channel 20. The second outer channel 26 is oriented substantially parallel to the main channel 12 and first outer channel 20. Second outer channel 26 has a first end and a second end with a third inlet 28 at the first end of the second outer channel 26, where the third inlet 28 is configured to flow a third fluid into the second outer channel 26 to a third outlet 29 at the second end of the second outer channel 26.

In devices with a first and second side outer channel, there is also a second-side plurality of microchannels 32 connecting the main channel 12 to the second outer channel 26 and oriented substantially perpendicular to the main channel and second outer channel. As with the first-side plurality of microchannels, sections of the microchannels may also be referred to herein as “migration channels”. The second-side microchannels 32 are in fluidic communication with the main channel and the second outer channel and, like the first-side plurality of microchannels, configured to establish a fluid chemogradient between the main channel and second outer channel (and/or between the first outer and second outer channels) when one or more chemo-modulatory fluids are flowed through one or more of the main channel, first outer channel, and/or second outer channel.

Similarly, two-sided devices can also include one or more second-side collection channels 46 located in between and oriented substantially parallel to both the main channel 12 and second outer channel 26 and in fluidic communication with the second-side plurality of microchannels 32. The one or more second-side collection channels can each have a first and second end, each second-side collection channel being in fluidic communication with a second flushing port 48 at the first end and each having an individual collection outlet 49 at the second end of each second-side collection channel 26. Each second-side collection channel is configured to flow a flushing fluid from the second flushing port 48 to the individual second-side collection outlet 49 such that any cells located in that second-side collection channel 46 are flushed by the flushing fluid to the individual collection outlet 49 for that second-side collection channel.

As discussed above, it is contemplated that the migratory regions, defined by the plurality of microchannels provide for a single-cell level migration. In other words, the microchannels allow about 1 cell to pass through, though more than one may be lined up within the channel (e.g., in “single file”). Thus, the main channel and outer channels have a larger width and height than the plurality of microchannels. In embodiments, the shape of the channels is substantially rectangular, with a rectangular cross-section (but it is contemplated that other geometries, such as circular, ovoid, trapezoid, etc. are also possible). In embodiments, the width dimension of any of the main channel and first and/or second outer channel is about 50 μm to 1000 μm, and the width of any of the first first-side or second-side collection channels is about 50 μm to 200 μm. In embodiments, the height of any channel that can be a larger cell flow channel (e.g., main channel, outer channel, or collection channel) is greater than the height of a microchannel in the plurality of microchannels such that cells squeeze/deform slightly to enter migratory microchannel. In embodiments, the height dimension of any of the main channel, first or second outer channel, and first-side or second-side collection channels is about 30 μm to 150 μm (e.g., about 50 μm). In embodiments each of the plurality of microchannels has a width of about 8 μm to 40 μm and a height of about 3 μm to 7 μm (e.g., 4 μm).

In embodiments, the length of the main channel, outer channel(s), and any collection channel(s) are roughly similar. The length of the device, and thus channels, in part determines how many cells can be passed through, evaluated, etc.. In embodiments, the main channel, first outer channel, second outer channel, first-side collection channels, and/or second-side collection channels have a length of about 10 mm to 60 mm.

Also, it should be noted that although both the chemo-modulatory supply channel and at least one cell flow channel may have dimensions in the micro-range, it is to be understood that the plurality of microchannels have a diameter substantially smaller than the supply and flow channels. At least one purpose of the microchannels is to allow slow migration of cells along the chemogradient between the supply and flow channels. In embodiments, the microchannels have a diameter sufficient to allow about one cell to flow (e.g., cells in a single-file line).

In embodiments, a device of the present disclosure includes from about 100 to 3500 (e.g., about 2500) microchannels per side of the device (e.g., about 2500 first-side microchannels and about 2500 second-side microchannels, for a total of about 5000 microchannels). The total number of microchannels depends on the size of the chip substrate, desired width of the microchannels, number of collection channels, etc., and variations on such design parameters are intended to be included within the scope of the present disclosure.

In embodiments, devices of the present disclosure, the channels of the devices are pre-coated with a protein or other layer to help cells maintain contact with the substrate in order to maintain cell viability. However, cells are not adhered statically to this coating layer, so they can still migrate within the device in response to the chemogradient. Suitable proteins for such coating include, but are not limited to collagen, poly-L-Lysine, poly-D-Lysine, and the like. For instance, in an embodiment, the channels are be pre-coated with collagen prior to loading of the cell-containing biological sample fluid.

The present disclosure also includes kits including devices of the present disclosure with instructions for use. In embodiment the kits can include any of the variations of the devices of the present disclosure described above, at least one chemo-modulatory fluid or instructions for preparing at least one chemo-modulatory fluid, and instructions for use of the device and the at least one chemo-modulatory fluid to separate target cells from a biological sample fluid. In embodiments, the chemo-modulatory fluid can be a chemo-attractant for the target cells, a chemo-repellent for non-target cells, or both, or vice versa, such as any of the list of chemo-modulatory compounds discussed in Table 1, above. In embodiments, the instructions explain how to introduce the chemo-modulatory fluid to create a chemogradient. In embodiments, the target cells are CTCs and the chemo-modulatory fluid is a chemo-attractant that attracts CTCs (e.g., FBS), a chemo-repellent that repels WBCs (e.g., Slit2), or a combination of both.

Also, in embodiments, the kit can include instructions for making and/or using at least two chemo-modulatory fluids and/or can include two or more chemo-modulatory fluids. In embodiments, the two or more chemo-modulatory fluids can include at least a first chemo-modulatory fluid that includes a chemo-attractant for a CTCs (e.g., FBS) and optionally a chemo-repellent for WBCs (e.g., Slit2). In embodiments, the kit includes a second chemo-modulatory fluid or instructions for preparing one, where the second chemo-modulatory fluid can include a chemo-repellent for WBC's (e.g., FMLP).

The kits of the present disclosure can include chemo-modulatory compositions and/or instructions for preparing a chemo-modulatory fluid compositions depending on the needed features of the chemo-modulatory fluid composition for the particular assay.

The kit can include instructions for loading the chemo-modulatory fluid in the main channel and the biological sample fluid in the first and/or second outer channels as shown in the top figure of FIG. 1C and/or instructions for loading the chemo-modulatory fluid(s) in one or more of the first or second outer channels (e.g., to create one or more chemogradients) and loading the biological sample fluid into the main channel (as illustrated in FIG. 1C, bottom).

Method of Use

The present disclosure further includes methods of using the devices of the present disclosure described herein to separate target cells from a sample and/or from non-target cells in a sample. In embodiments, methods include using the devices of the present disclosure to separate CTCs from a biological sample. In embodiments, methods also include separating CTCs from white blood cells in the sample. In further embodiments, the methods include separating CTCs in the sample into subtypes of CTCs (e.g., invasive, non-invasive, etc.) based on the migratory profile of the CTCs. For instance, as demonstrated in the examples below, in some instances invasive CTCs migrate faster and further in the devices of the present disclosure (e.g., toward the chemo-stimulant composition) than non-invasive CTCs.

In general, methods of the present disclosure for separating target cells in a biological sample can include introducing a chemo-modulatory fluid into the main channel or first or second outer channel of the device of the present disclosure to create a chemogradient and introducing a biological fluid sample into the other of the main channel of outer channel. In embodiments, the biological fluid sample with cells can be added to the device first (either via the first inlet/main channel or via the second/third inlets of the first and second outer channels). In embodiments, the when the biological fluid sample is added, it is initially flowed at a rate of about 1 to 10 μL/min (e.g., 5 μL/min) for a period of about 1-5 min to introduce cells to the device. In embodiments, the device with fluid cell sample added is incubated for about 30 to 60 min to allow the cells to become established in the device. In embodiments, the concentration of cells in the biological sample fluid can be about 1×10⁵ to 10×10⁵ cells/mL (e.g., about 5×10⁵ cells/mL). In embodiments, after loading of the cell sample fluid, a fluid free of any chemo-modulatory compound (e.g., cell culture medium) can be introduced into the channel(s) carrying the biological fluid sample, and chemo-modulatory fluid can be added to the other channel (whichever channel, main or outer, is not flowing the biological sample fluid). The culture medium/biological sample fluid and the chemo-modulatory fluid can be flowed through the device at a rate of about 0.05-0.5 μL/min such as, about 01. μL/min.

In embodiments, the device can be incubated for a time during which cells are allowed to migrate through the chemogradient in the device. In embodiments, the device is incubated for about 6-24 hours. In embodiments, cells can be observed and/or collected at various time periods by flowing a flushing fluid (e.g., non-chemo-modulatory culture medium, or biocompatible carrier fluid, etc.) through one or more of the collection channels, such that cells located in the collection channels can be collected at the collection channel outlets. In embodiments where the device microchannels include a protein coating (e.g., collagen) to help cells maintain contact/interaction with the substrate, the flushing fluid can include a compound (e.g., trypsin-EDTA) to help release cells from contact/interactions with the protein coating. Cells collected at the collection channels furthest from the channel flowing the chemo-modulatory fluid at the earliest collection time would be the cells with the highest migration rate. Cells that do not migrate from the channel flowing the biological sample fluid or that have not migrated very far (e.g., only to the first collection channel) and/or have migrated slow and are collected at a later time point, are cells with a lower migration rate. At some time periods, a flushing fluid or other suitable fluid may be run through one or more of the main or outer channel in order to collect all remaining cells.

In some embodiments, if only a single side device is used, the method can include introducing a chemo-modulatory fluid in the first inlet of the main channel or the second inlet of the first outer channel, such that the chemo-modulatory fluid flows in the device and establishes a chemogradient between the main channel and the first outer channel of the device. For instance, the chemo-modulatory fluid can be introduced in the first inlet of the main channel such that a chemogradient is established moving out from the main channel (e.g., highest concentration of chemo-modulatory compound closest to the main channel). But in another embodiment, the chemo-modulatory fluid can be introduced in the first outer channel of the device, creating a chemogradient with higher concentration near the first outer channel. A biological sample fluid is introduced (via an inlet) and flowed into the other of the main channel or first side channel that is not flowing the chemo-modulatory fluid. Thus, whichever of the main or outer channel is going to flow the chemo-modulatory fluid, the other flows the biological sample fluid.

The method then includes allowing and detecting migration of target cells and non-target cells in the biological sample fluid in response to the chemogradient. Depending on the type of chemo-modulatory fluid used, the target cells in the sample move toward or away from the chemo-modulatory fluid in response to the chemogradient, non-target cells in the sample migrate toward or away from the chemo-modulatory fluid in response to the chemogradient, or both. Regardless, the movement of the target cells is distinguishable from the movement of the non-target cells based on one or more of speed and distance of migration of the cells across the chemogradient in the device via the plurality of microchannels. In embodiments, after introduction of the chemo-modulatory fluid and the sample biological fluid into the device, the device may be incubated to allow migration of the cells.

After various time points for migration of the cells, the cells can be flushed from the device to separate migrated cells (e.g., target cells) from non-migrated (or oppositely migrated) cells (e.g., non-target cells). In embodiments a flushing fluid can be used to flush channels of the device to remove cells from flushed channels. In devices with one or more collection channels, the collection channels can be selectively flushed with a flushing fluid at various time points to flush the cells from the collection channels to determine migratory distance, speed, and the like. The method can include introducing and flowing a flushing fluid from the first flushing port through the one or more first-side collection channels such that any target or non-target cells located in the one or more first-side collection channels between the main channel and the first outer channel are flushed by the flushing fluid to the individual collection outlets and are separated into different collection outlets based on distance of migration within the device.

In other embodiments, a 2-sided device can be used, and methods can include the methods above, except that when a chemo-modulatory fluid is introduced in the main channel, the biological sample fluid is introduced in to each of the first and second outer channels. If the biological sample fluid is introduced into the main channel, the same chemo-modulatory fluid can be introduced and flowed through each of the first and second outer channels, or two difference chemo-modulatory fluids can be used and introduced into different outer channels. In such an embodiment, 2 different chemogradients (or a least a 2-sided chemogradient) can be created, with the most neutral concentration in the main channel, and a chemogradient on either side. In an embodiment, a chemo attractant for the target cells can used in the chemo-modulatory fluid introduced at the second inlet for the first outer channel and a chemo-repellent for the target cells can be included in as second chemo-modulatory fluid introduced at the third inlet for the second outer channel.

Thus a chemo-modulatory fluid can be introduced in either the first inlet of the main channel or in both of the second and third inlets of the first and second outer channels, respectively, such that the chemo-modulatory fluid flows in the device and establishes a chemogradient between the main channel and the first and second outer channels of the device. A biological sample fluid including target cells can be introduced and flowed into the other of the main or first and second outer channels that isn't flowing the chemo-modulatory fluid. The device can optionally be incubated for various time periods to allow for cell migration. The method includes allowing and detecting migration of target cells and non-target cells in the biological sample fluid in response to the chemogradient, such that target cells in the sample move toward or away from the chemo-modulatory fluid in response to the chemogradient, non-target cells in the sample migrate toward or away from the chemo-modulatory fluid in response to the chemogradient, or both, and wherein the movement of the target cells is distinguishable from the movement of the non-target cells based on one or more of speed and distance of migration of the cells across the chemogradient in the device via the plurality of microchannels. Non-target cells may also remain neutral to a chemogradient (particularly if it only includes a chemo-attractant for the target cells), but this will still be distinguishable from a movement of the target cells within the device towards or away from a particular chemogradient. Then a flushing fluid is introduced and flowed (either through the main and outer channels of the device or, if present, through one or more collection channels). The flushing fluid can be introduced via one or both of the first and second flushing ports for the one or more first side or second side collection channels. Thus, any cells (target cells or non-target cells) located in the one or more collection channels gets flushed to one or more collection outlet.

As mentioned above, in some embodiments of method of the present disclosure, the chemo-modulatory fluid is the first fluid and is flowed from the first inlet through the main channel such that a chemogradient is established between the main channel and each of the first and second outer channels. In such embodiments, the biological sample fluid can comprise a first and second biological sample fluid, wherein the first and second biological sample fluids are the same or different, the first biological sample fluid is flowed from the second inlet through the first outer channel and the second biological sample fluid is flowed from the third inlet through the second outer channel, such that target cells in each of the first and second sample fluids move toward or away from the main channel via the first and second plurality of micro channels in response to the chemogradient.

In embodiments, the chemo-modulatory fluid is a chemo-attractant for the target cells and optionally also includes a chemo-repellent for non-target cells, such that the target cells move toward the chemo-modulatory fluid in the main channel in response to the chemogradient. The method can further include sub-typing the target cells based on the speed and distance migrated from the first or second outer channel toward the main channel. In embodiments, as discussed above, the target cells are circulating tumor cells (CTCs), the non-target cells are white blood cells (WBC's), and the chemo-modulatory fluid includes as chemo-attractant for CTC's and a chemo-repellent for WBC's.

In other embodiments of methods of using the devices of the present disclosure, the biological sample fluid is the first fluid and is flowed from the first inlet through the main channel, the chemo-modulatory fluid includes a first and second chemo-modulatory fluid and the first and second chemo-modulatory fluids can be the same or different. The first chemo-modulatory fluid is flowed from the second inlet through the first outer channel such that a first chemogradient is established between the first outer channel and the main channel, and the second chemo-modulatory fluid is flowed from the third inlet through the second outer channel, such that a second chemogradient is established between the first outer channel and the main channel. Target cells in the biological sample fluid move from the main channel toward or away from the first and second outer channels via the first and second plurality of micro channels in response to the first and second chemogradients.

In embodiments where the first and second chemo-modulatory fluids are the same and include a chemo-attractant for the target cells and optionally include a chemo-repellent for non-target cells, the target cells move toward the first and second chemo-modulatory fluids in the first and second outer channels in response to the first and second chemogradients. Such methods can further include sub-typing the target cells based on the speed and distance migrated toward the first or second outer channel. In other embodiments, the first and second chemo-modulatory fluids are different, where the first chemo-modulatory fluid includes a chemo-attractant for the target cells and optionally a chemo-repellent for non-target cells, and where the second chemo-modulatory fluid comprises a chemo-attractant for non-target cells. The target cells move toward the first outer channel in response to the first chemogradient and the non-target cells remain in the main channel or move toward the second outer channel in response to the second chemogradient. For instance, in embodiments, the target cells are CTCs and the non-target cells are WBC's, the first chemo-modulatory fluid includes a chemo-attractant for CTC's and optionally a chemo-repellent for WBC's. The second chemo-modulatory fluid can also include a chemo-attractant for WBC's.

In embodiments the biological sample fluid is a fluid prepared from a biological sample obtained from a patient. In embodiments, it is a processed blood sample, such as a blood or other fluid sample processed to remove red blood cells, or to remove most cells other than target cells with some WBC contamination. In embodiments the biological sample fluid is a blood or other biological fluid sample that has been processed by other methods to pre-enrich the target cells, such as CTCs. In embodiments, the biological sample fluid has been processed through a previously described cell separation method designed to remove a majority of white blood cells from a sample containing target cells (e.g., CTCs) and white blood cells, without labeling the target cells. Other cell separation methods that can be used to pre-enrich CTCs can include methods like iFCS (integrated ferrohydrodynamic cell separation) described in Zhao et al., (Lab on a Chip 2019, 19 (10), 1860-76), and U.S. patent application Ser. No. 16/406,440 (publication No. US 2019/0262836), which are hereby incorporated by reference in their entireties.

As will be appreciated by a skilled artisan the devices of the present disclosure can be run in different modes depending on which fluid is flowed in which channel. Also, difference cells can be manipulated/modulated depending on the choice of chemo-modulatory agent, how they are combined, and where the chemogradient is established in the device. As such variations of the above-described devices, kits, and methods exist and are intended to be included in the scope of the application.

Additional details regarding the methods, compositions, and organisms of the present disclosure are provided in the Examples below. The specific examples below are to be construed as merely illustrative, and not !imitative of the remainder of the disclosure in any way whatsoever. Without further elaboration, it is believed that one skilled in the art can, based on the description herein, utilize the present disclosure to its fullest extent. All publications recited herein are hereby incorporated by reference in their entirety.

It should be emphasized that the embodiments of the present disclosure, particularly, any “preferred” embodiments, are merely possible examples of the implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure, and protected by the following claims.

The following examples are put forth so as to provide those of ordinary skill in the art with a complete disclosure and description of how to perform the methods and use the compositions and compounds disclosed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, the term “about” can include traditional rounding according to significant figures of the numerical value. In addition, the phrase “about ‘x’ to ‘y’” includes “about ‘x’ to about ‘y’”.

### EXAMPLES

Now having described the embodiments of the disclosure, in general, the examples describe some additional embodiments. While embodiments of the present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

### Example 1

### Microfluidic Assay for Isolation and Characterization of Circulating Tumor Cells Introduction

In 2020, approximately 276,480 women will be diagnosed with breast cancer while about 191,930 men will be diagnosed with prostate cancer.¹ Although the death rate will decrease this year, about 31% of the cancer patients will still lose their lives.¹ Cancer metastasis, the spread of tumor cells from the primary tumor to another part of the body, represents a major cause of cancer-related death.² Circulating tumor cells (CTCs) play an important role during cancer metastasis and have significant implications for basic and translational research of cancer.³⁻⁴ CTCs are heterogeneous due to the epithelial-to-mesenchymal transition (EMT), and they have different intrinsic properties, such as invasion, migration, and proliferation. It is believed that only a small portion of CTCs could extravasate and develop a secondary tumor. The isolation and characterization of these metastasis carriers would enable us to predict the probability for a patient to develop metastasis, evaluate the outcome of treatment, and avoid cancer recurrence. However, the major limitation of research with CTCs is their very low occurrence in the blood, at only about 0 to 100 CTCs per milliliter of whole blood. As a result, development of innovative technologies that can enrich and characterize viable CTCs with high purity and high recovery rate could help researchers study the migratory behaviors of CTCs, understand metastasis, and apply this technology in the manipulation of patient-derived samples.

Current CTC isolation techniques could be classified into two categories: label-free and label-based. Label-free techniques rely on the physical properties of CTCs, such as size⁵, density⁶, and deformability⁷, while label-based techniques target biological markers on the cell surface⁸⁻⁹. However, many studies found that CTCs are rare and occur in heterogeneous populations of different subtypes,^{4, 10} which make it difficult to simultaneously maintain a high purity and high recovery rate. The state-of-art CTC isolation technique, iFCS¹⁰, can achieve a 99.1% recovery rate, 12 mL/h throughput, and remove more than 99.992% WBCs without affecting cell viability. However, there were still about 500 WBCs left after processing 1 milliliter of blood, which will introduce high background noise in the downstream CTC analyzations, especially in single-cell sequencing. Moreover, the studies of the relationship between CTC and metastasis were constrained to the quantification of isolated CTCs and some geomatic analysis. There is no known study of the migration of CTC in vitro due to the low occurrence and technical limitations.

The conventional approaches to study cancer invasion and motility in vitro are wound healing and Boyden chamber assays¹¹⁻¹². The wound healing assays were limited by the low reproducibility and lower recovery rate due to difficulties with isolation of migratory cells from less migratory cells. Boyden chamber assays can perform migration assays in a large cell population, but it can only maintain a high concentration gradient for several hours, and the bulky setup makes it impossible to track induvial cells. These limitations preclude the use of these approaches in a CTC migration assay or any migration assay for rare target cells. The microfluidic platform of the present disclosure provides a stage to precisely control and study the cancer metastasis on-chip and under optimized conditions. 2D migration assays rely on different channel geometries to direct cell movement in a confined area,¹³⁻¹⁵ while the 3D migration studies use microfluidic chips filled with hydrogel to simulate the local microenvironment of a tumor¹⁶⁻¹⁸. However, microfluidic approaches that study collective or single-cell migratory behaviors face challenges with retrieving cells from the microfluidic channel, especially for cells that easily become trapped in the confined space. In addition, current 2D techniques can only categorize cancer cells into two subtypes including migratory and non-migratory cells, while overlooking cells in the migration channels. Moreover, there were no consensus parameters, such as physical properties and biological markers, that could illustrate cancer migratory behaviors.

The devices, kits and methods of the present disclosure overcome these challenges and limitations. In the present example, a single-cell migration chip (sometimes referred to in the present example as “MChip”) was provided, which allowed the study of migratory behaviors of CTCs on the single-cell level. The device also facilitated classification of the cells into 9 subtypes based on the migration distance and allowed retrieval of each of these sub-populations without affecting their viability and proliferation properties. The MChip was designed and optimized to geometrically mimic the confined spaces of the endothelial layer of a blood vessel through which CTCs must deform and squeeze out to invade the distant tissue¹⁹. In this embodiment of a microfluidic device of the present disclosure, three collection channels were included along the pathway of single-cell migration channels, enabling easy identification and retrieval of subtypes for downstream analysis. MChip, was tested with eight different cancer cell lines, including one triple-negative breast cancer cell line, and optimized for single or combined parameters to predict the migratory and motility characteristics of cancer cells. The average migration distance was proportional to the aspect ratio, and there was a negative correlation between CD44 marker expression level and migration distance. The combination of aspect ratio and vimentin maker expression level showed a strong relationship with cell migratory behaviors. MChip can further separate/subtype CTCs enriched from iFCS based on the different migration directions controlled by combinations of chemo-attractant or chemo-repellent compounds. The present example demonstrates that the devices and methods of the present disclosure were able to remove more than 99.6% WBCs from a biological fluid sample, leaving only about 2 WBCs after processing 1 milliliter of whole blood. Thus, the devices, kits, and methods of the present disclosure can also be used in conjunction with existing CTC separation technologies to further enrich, separate, and characterize the CTCs in a sample.

### Materials and Methods

MChip Flow Profile and Concentration Gradient Simulation

The computational fluid dynamics and transport of diluted species simulation were conducted using COMSOL Multiphysics (Version 5.5, COMSOL Inc., Burlington, MA). A 2D model was developed to study the incompressible laminar flow with Navier-Stokes equations and transport of diluted species with the stationary mass-balance equation. Cell culture medium properties (density=1.007 g/cm³, viscosity=0.94 mPa·s) and a diffusion coefficient of 61 μm²/s (albumin in FBS) were used to model the flow profile and concentration of FBS in the microchannel. The velocity in the main channels (normal inflow velocity=0.1 μL/min) and migration channels were modeled first, including extra fine mesh of 611,314 elements to ensure that the results were independent of the discretization of the computational domain. Then, the concentration distribution of FBS was simulated based on the flow profile of step 1. The initial concentration across the whole domains was 0%.

MChip Design and Fabrication

The embodiment of the microfluidic device of the present example (MChip) contains nine flow channels (two cell loading channels (e.g., side channels), one serum loading channel (main channel), and six collection channels), and eight migration regions (4 on each side, first-side and second-side plurality of microchannels), for a total of 5000 migration channels (2500 channels in one side). The multiple heights for the main channel (50 μm) and migration region (4 μm) were fabricated on a chip substrate with two photomasks. Briefly, the 4 μm migration region was firstly coated onto the silicon wafer (WaferPro, Santa Clara, Calif.) with SU-8 3005 photoresist (MicroChem, Westborough, Mass.). Then the 50 μm main channels were added with SU-8 2025 (MicroChem, Westborough, MA). The channel heights were measured by a profilometer (Veeco Instruments, Chadds Ford, Pa.). Based on the soft lithography techniques, polydimethylsiloxane (Dow Corning, Midland, Mich.) were poured onto the silicon wafer, baked at 80° C. for 2 hours, and bond with a glass slide to give the microfluidic device.

Cell Culture

Cancer cell lines (ATCC, Manassas, Va.) including one prostate cancer cell line (PC-3), three non-small cell lung cancer (NSCLC) cell lines (H1299, H3122, and A549), and four breast cancer cell lines (MDA-MB-231, MCF7, HCC1806, and HCC70) were used in this study. The cell lines were cultured following the manufacture's protocol.

Device Disinfection and Coating

Microfluidic devices were immersed into 70% Ethanol and disinfected under UV light for 12 hours. Extra ethanol was removed by air dry in the biosafety cabinet. Microfluidic channels were coated with 50 μg/mL type 1 collagen solution (Advanced Biomatrix, San Diego, Calif.) following the manufacturer's recommended protocol. The coated devices were stored at 4° C. before the experiment.

Cell Loading of the Migration Assay

The microfluidic device was pre-filled with FBS-free cell culture medium and incubated at 37° C. for 1 hour. All of the channel inlet/outlet, except cell loading inlets (top/bottom channel #1) and middle channel outlet, are sealed with tape before cell loading. Cancer cells were collected from culture plates with 0.25% Trypsin-EDTA (ThermoFisher, Waltham, Mass.) and pelleted by centrifugation at 500 g for 5 minutes. Then, the cells were re-suspended in FBS-free culture medium, DMEM or RPMI (ThermoFisher, Waltham, Mass.), to a concentration of 5×10⁵ cells/mL. 50 μL of cell suspension was pipetted into the cell loading channels. One syringe pump (Chemyx, Stafford, Tex.) was connected to the middle channel outlet and the flow rate was 5 μL/min. When all entrances of migration channels were filled with cancer cells, the pump was stopped and the cell suspension was replaced with FBS-free cell culture medium. The device was then placed in the incubator (37° C., 5% CO₂) for 40 min to ensure the cell adhesion to the glass slide. Then, the tape/covers were removed from the cell loading outlet and middle inlet, and these inlets were connected with syringe pumps. The FBS-free medium flowed into the cell loading channel while the middle channel was filled with medium containing 10% FBS. The flow rates for 3 channels were 0.1 μL/min. The entire chip was placed in the cell culture incubator (37° C., 5% CO2) for about 24 hours.

Cell Loading of the Purification Assay

WBCs and MDA-MB-231 cancer cells were mixed at a 1:1 ratio in FBS-free DMEM medium and loaded to the top edge of the middle/main channel. The positions of these cells were determined by an optimized flow rate and open/close of channel inlets/outlets. Briefly, the microfluidic device was filled with cell culture medium and incubator at 37° C. for one hour. Sealed all the inlet/outlets except cell loading inlet (middle/main channel) and channel #1 outlet (top). 50 μL cell mixture was loaded into cell loading inlet and the cell alignment was achieved using a syringe pump (5 μL/min) that connected to the channel #1 outlet (top). Then replaced the cell suspension with FBS-free medium and incubated for 30 minutes. The FBS-free medium flowed into the middle channel, the top channel (first outer channel) #1 was filled with medium containing 10% FBS, and the bottom channel (second outer channel) +1 was filled with FBS-free medium containing FMLP (200 ng/mL). The flow rates for 3 channels were 0.1 μL/min. The cells were incubated at 37° C. for 24 hours.

Cell Retrieval and Long-Term Proliferation Assay

After the migration assay, the cell culture medium was replaced with 0.25% Trypsin-EDTA and incubator for 5 minutes at 37° C. Tapes were removed and Teflon tubes were connected to the collection channels. Cells will be flushed out by cell culture medium and collected onto the 48-well plate (Corning, Corning, N.Y.). The medium will be removed by centrifugation (200 g, 5 min) and replaced with 1 mL cell culture medium (with FBS). The medium was refreshed every 24 hours during the first 3 days. The number of cells and cellular morphology were inspected at 0 hour and 72 hours.

Characterization of Cell Viability During the Cell Migration Assay

The cell viability assays were performed after 6 hours', 12 hours', and 18 hours' migration using the Live/Dead viability/cytotoxicity kit (ThermoFisher, Waltham, Mass.). Briefly, the medium in the microfluidic channels was replaced with a working solution (2 μM calcein-AM and 4 μM propidium iodide (PI) in D-PBS) for 35 minutes at room temperature. After the solution was removed and washed twice with PBS, cells were observed and counted under an inverted microscope (Carl Zeiss, Germany).

Human Subject Statement and Sample Processing with iFCS

The blood sample was drawn from multiple healthy donors in compliance with the applicable federal, state, and institutional policies and procedures in the United States. Healthy human blood samples were obtained from the Clinical and Translational Research Unit of the University of Georgia with informed consent according to an IRB-approved protocol. The collected blood samples were stored in vacutainer K₂-EDTA (BD, Franklin Lakes, N.J.). Complete blood count (CBC) reported was obtained to determine the number & subtypes of WBCs. The blood processing with iFCS was discussed in our previous work (Zhao, et al., Lab on a Chip 2019, 19 (10), 1860-76, incorporated by reference above).¹⁰

Immunofluorescence Staining

After the migration assay, cells in the microfluidic device were fixed with 4% (w/v) PFA (Santa Cruz Biotechnology, Dallas, Tex.) for 10 minutes and subsequently permeabilized with 0.1% (v/v) Triton X-100 (Alfa Aesar, Haverhill, Mass.) in PBS for 10 minutes. Blocking buffer (Santa Cruz Biotechnology, Dallas, Tex.) was applied for 30 minutes to block the nonspecific binding sites of cells. Cells were stained with primary antibodies including anti-EpCAM, anti-CD44, and anti-vimentin (Santa Cruz Biotechnology, Dallas, Tex.) at 4° C. overnight. Then, stained cells were washed with PBS and covered with DAPI-Fluoromount (Electron Microscopy Sciences, Hatfield, Pa.) before imaging.

CellTracker Labeling

MDA-MB-231 and MCF7 were labeled with either CellTracker Green CMFDA dye or Red CMTPX dye (ThermoFisher, Waltham, MA) following the manufacturer's protocol. Briefly, the cell culture medium was removed when the cell confluent reached ˜70% and washed with PBS. The cells were incubated with 5 mL DMEM containing 0.5 μM dyes at 37° C. for 30 minutes and subsequently washed twice with PBS.

Image Acquisition

After staining, the MChip was imaged using an inverted microscope. 2.5× objective was used to take fluorescence images of the nucleus (DAPI) for analyzation of cell final position. 10× objective was used to take both phase-contrast and fluorescence images of cells for the analyzation of migration distance, morphologies (aspect ratio, circularity, solidity, and area), and marker expression levels (gray value of fluorescence intensities). All the 10× fluorescence images were of the same exposure time.

Cell Counting and Characterization Program

The final positions, cell morphologies, and the marker expression level were obtained using ImageJ software.

### Results and Discussion

Device Description

The microfluidic assay using an embodiment of the device of the present disclosure (referred to in the present example as “migration-based microfluidic platform or “MChip”) was developed to isolate and purify circulating tumor cells (CTCs), and simultaneously characterize and quantify the migratory properties of the CTCs. In this strategy, the microfluidic platform includes cell loading channel(s) (which can be the main channel or the first and/or second outer channel, depending on use), chemo-attractant channel(s) (e.g. serum) for carrying the chemo-modulator (which can also be the main channel or the firs and/or second outer channel(s), depending on mode of operation), one or more collection channels, and migration channels (e.g., plurality of microchannels). In the mode of operation shown in FIG. 1A, all the cells were injected in the cell loading channel and positioned at the same starting point, which is achieved by the height difference between loading and migration channels. Under the effect of chemo-attractant, cells deformed and squeezed into the confined migration channels (running perpendicularly to the cell loading channels) in an effort to migrate to the area with a higher concentration of chemo-attractant. Thus, the cells in the MChip can be classified into at least two categories: as non-migratory if the cell stayed in the loading channel and didn't enter the migration channel, or as migratory if they could deform and reach to the migration channels.

The migratory cells could be further subdivided into several types according to the final position they were located (e.g., how far they migrated within the chemogradient toward the chemo-attractant). Furthermore, cells could be retrieved from each channel (e.g., main, outer, or collection channels) for the downstream analyzation. Based on this principle, the PDMS based microfluidic device was developed (an example of which is illustrated in the images of FIG. 1B) using standard photolithography and soft lithography. FIG. 1C provides schematic drawings of embodiments of the microfluidic structure and two different modes of operation illustrating methods of use of the present disclosure. Since a majority of isolated CTCs are larger than 5 μm,¹⁰ the migration channel was designed with a height of 5 μm and a width of 30 μm so that all the cells could be trapped near the entrance of migration channel. Conventional migration assays suffer from difficulties in retrieving cells in the migration path, which leads to a lower yield of cells. The embodiment of the present device illustrated in the present example incorporated 6 collection channels (3 on each side) into the migration pathway so that migratory cells could be easily collected and classified. Two cell loading channels, 5000 migration units (e.g., plurality of microchannels) (2500 on each side), and one chemo-attractant channel enlarged the capacity of MChip, so at least 5000 cells could be simultaneously studied. The images of FIG. 1D illustrate a large number of cells at different migration points within the device. This design provides a stage to correlate the cell migration properties to EMT markers and physical properties, isolate cancer cells based their functional (e.g., migratory) properties instead of size or biological markers, and provides researchers a super-purified CTC sample.

Fluidic and Concentration Gradient Simulation in the MChip for Cell Migration.

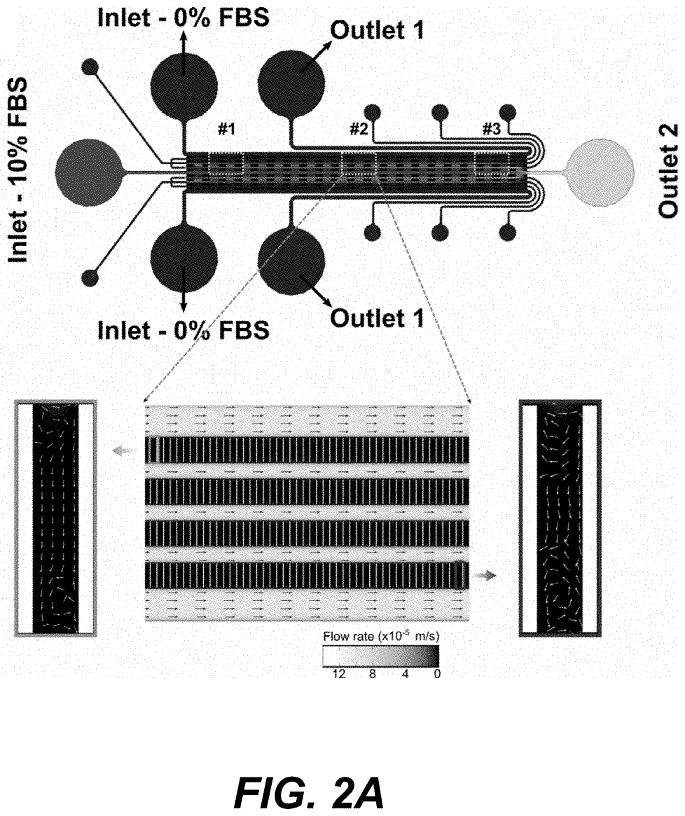

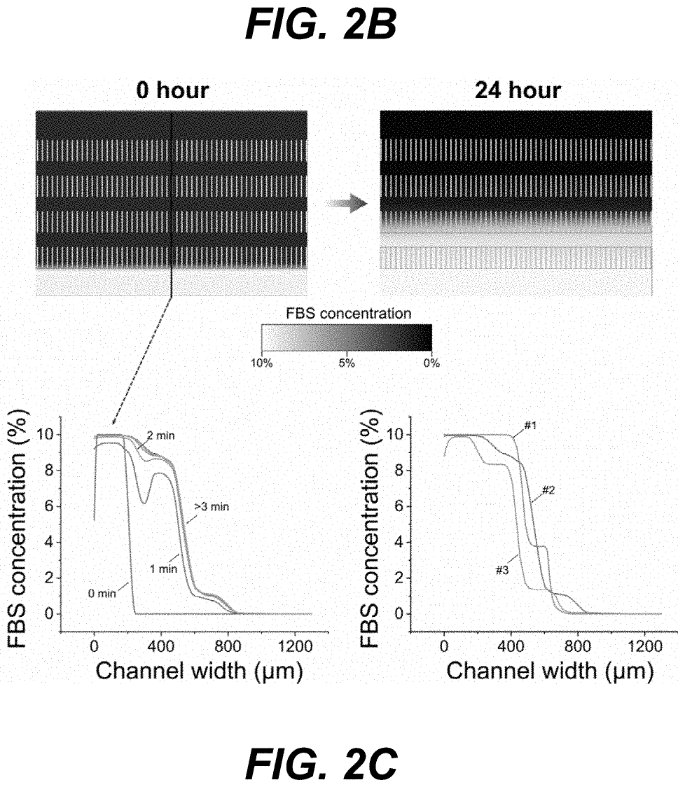

The migratory behavior of cancer cells is related to the concentration of chemo-modulatory compounds (e.g., chemokines, such as, but not limited to FBS) which could (in the case of a chemo-attractant) induce cells to move from the area with low concentration to the place with high concentration²⁰. To predict the local micro-environment and optimize the experimental parameters (flow rate and working time), a 2D computation fluidic dynamics model was developed to simulate the flow profile in the MChip under continuous flow and study concentration distribution across the channel over time. In the present example, as illustrated in FIG. 2A, the concentration difference in the channel was generated by infusing cell culture medium with 10% FBS into the middle channel while the cell loading channels (top/bottom channel #1 in FIG. 2A), were loaded with FBS-free cell culture medium. Flow rates for each inlet were set to 0.1 μL/min to refresh the medium and maintain cell viability. Interestingly, the flow rates in the migration channels (FIG. 2A, left and right zoomed images of the insert) were not zero and it could maintain a relatively suitable environment for cells. As shown in FIG. 2B, the concentration of the FBS increased from the cell loading channel to the middle channel under the influence of flow rate and diffusion. The concentration gradient (chemogradient) quickly stabilized within 5 minutes and had no significant change within 24 hours (FIGS. 2B-2C). Although the gradients slightly varied from start to the end of the channel (#1, #2 and #3 observation window from FIG. 2A) due to the flow profile difference, the migration direction was the same as predicted (FIG. 2C, right). The simulation results were experimentally verified using a fluorescence dye (Rhodamine B) at a flow rate of 0.1 μL/min. The fluorescence intensity of the middle area (top part) was measured and plotted at 0 hour and 24 hours, which indicated that the concentration gradient was successfully produced and agreed with simulation results (data not shown). The signal fluctuations were contributed by the autofluorescence of PDMS material. The concentration gradient was close to zero between the middle channel and the third collection channel (one closest to main channel), thus we believed that migratory cells would change its ‘middle-directed’ tendency in the third collection channel and reduce migration speed.

Validation of MChip with Cancer Cell Line.

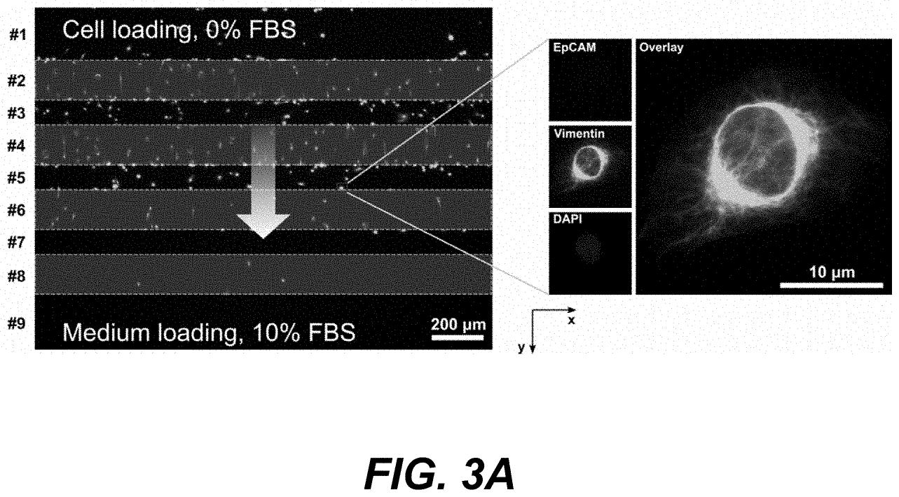

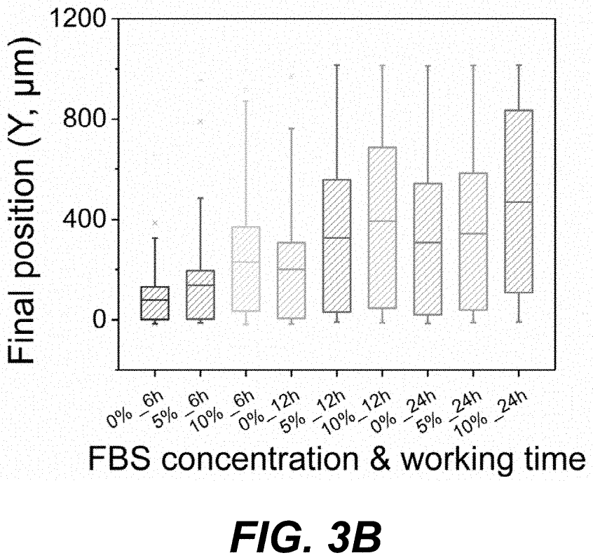

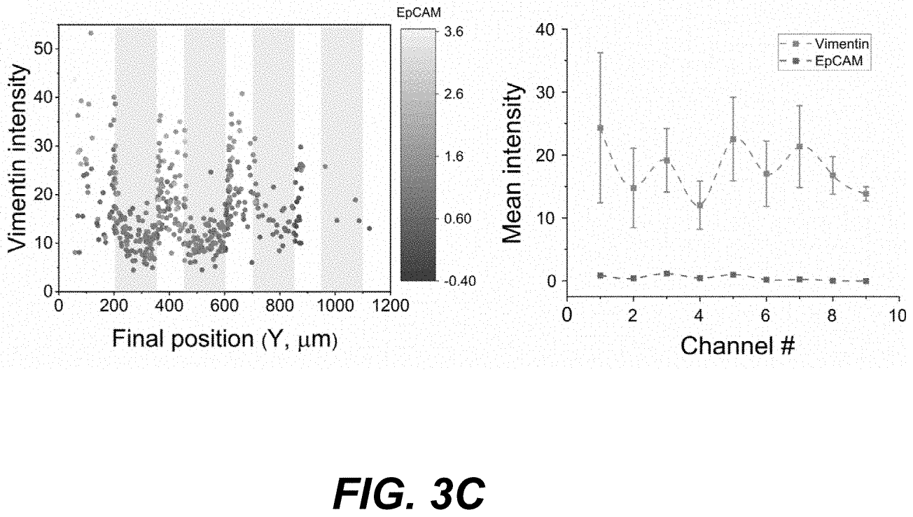

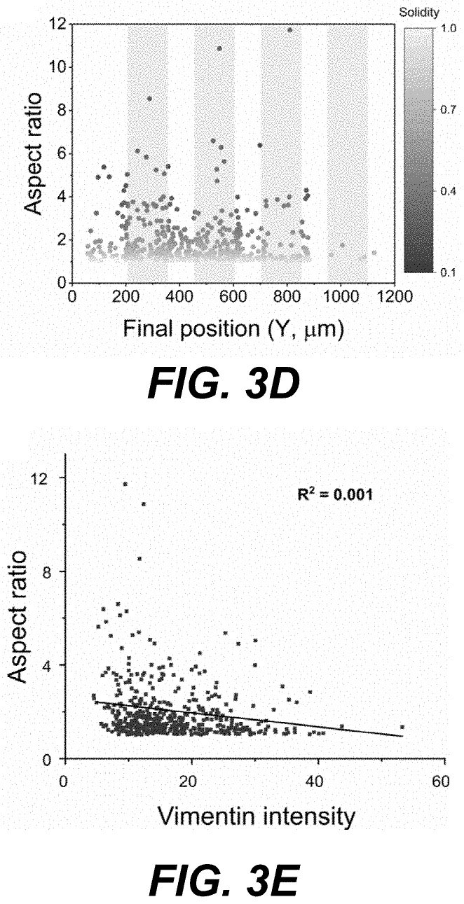

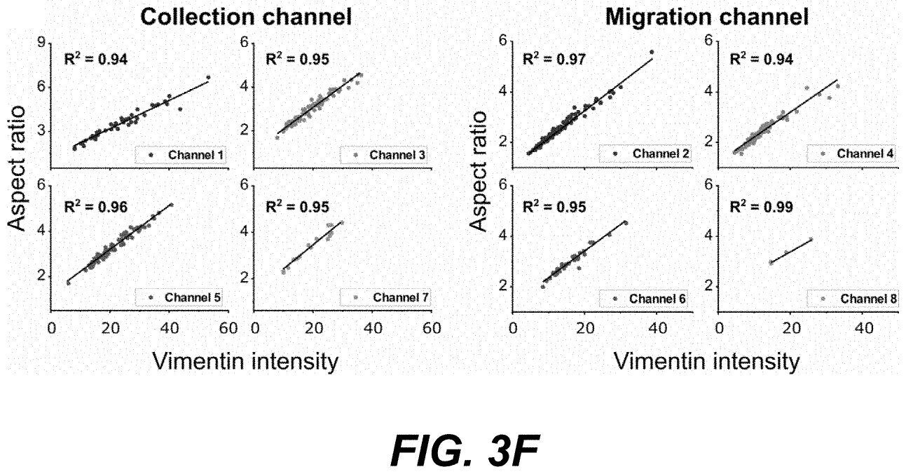

Using the optimized operation parameters and channel geometry established above, cancer cell migration was studied in MChip prototype devices using the breast cancer cell line, MDA-MB-231, to confirm the prediction of the simulation result. A typical migration assay can be visualized in FIG. 3A, in which -5000 cancer cells were loaded (channel #1) and positioned at the entrance of the first migration area (plurality of micro channels #2). FBS-free medium in the cell loading channels and 10% FBS medium in the middle channel (main channel #9) were simultaneously injected into each inlet. The MChip platform was placed in the incubator (37° C., 5% CO₂) to maintain cell viability. Migratory cells deformed and squeezed into the migration channel, reached the first collection channel, and kept moving to the next migration channel, leading the migration to continue until they reached to channel #9 of highest FBS concentration. While some of the non-migratory cells did squeezing into the confined space and moved a short distance, they did not move as far as the highly-migratory cells. After 24 hours' migration, information about the final position of the cells, physical properties and phenotype were extracted using immunofluorescence staining (FIG. 3A, right).

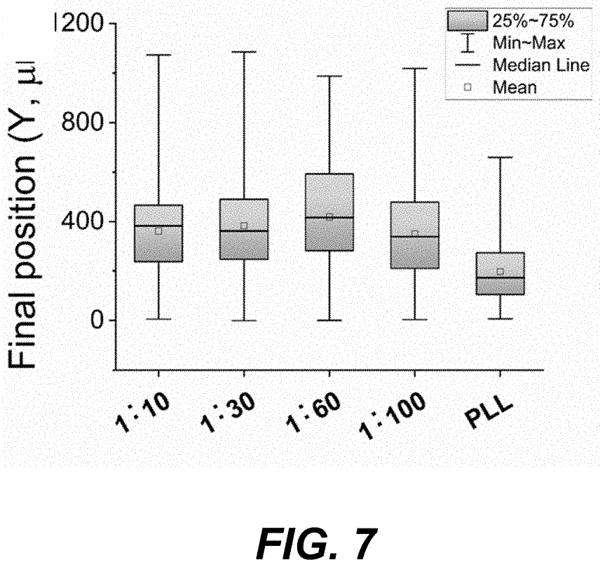

Since migration time^{14, 21} and FBS concentration were key parameters that determined the final position of individual cells, a series experiment were conducted to test the influence of different FBS concentrations (0%, 5%, and 10%) and various working time (6 hr, 12 hr, and 24 hr). As shown in FIG. 3B, final positions of cells were proportional to the FBS concentration and working time. Thus, optimized parameters, 10% FBS in the medium loading channel (#9) and 24 hours, would induce an abundance of migratory cells for the downstream analyzation. Thus, these parameters were chosen for application in the following experiments. It is noteworthy that cell migratory behaviors were also determined by the glass coating method, for example, coating using different concentrations of collagen and poly-L-Lysine (FIG. 7). The solution of 50 pg/mL collagen gave the largest average migration distance.

Characterization of Migratory and Non-Migratory Cell Phenotypes.

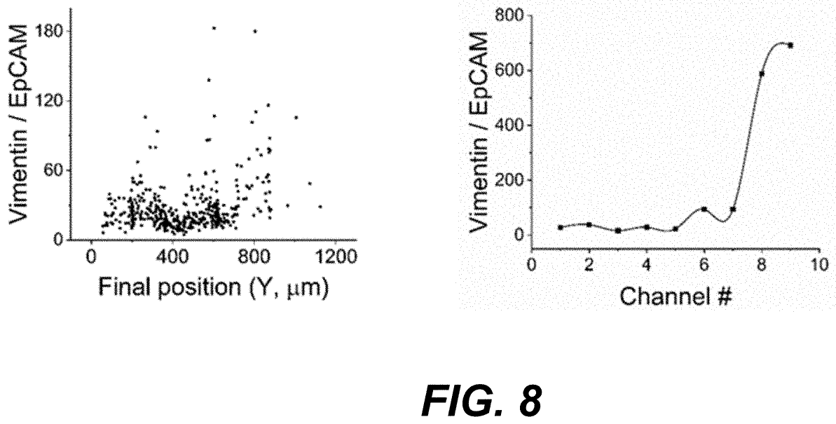

To understand the underlying causes of migration differences, the phenotypes of non-migratory and migratory MDA-MB-231 cancer cells were analyzed in MChip. The phenotypes could be characterized using expression level of EMT marker, including epithelial marker EpCAM and mesenchymal marker vimentin. EpCAM (epithelial cell adhesion molecule) is widely used as a marker of carcinoma and is typically overexpressed in different kinds of epithelial cancers.²² Epithelial cancers, such as MCF-10A,²³ often have limited migratory ability. Vimentin is a major component of intermediate filament family of proteins and plays an important role in maintaining cellular integrity, resisting stress and cell migration.²⁴ Vimentin could promote the formation of pressure-based blebs during cell migration by the interactions between nucleoskeleton and cell membrane in the cell.²⁵⁻²⁷ The graphs in FIG. 3C show the EpCAM and vimentin intensities in the microfluidic channels after 24 hour's migration. It is noteworthy that signal oscillations happened along the migration direction (FIG. 3C, left): average vimentin intensity decreased when the cells were in the migration channels (#2, #4, #6 and #8, the shaded rectangular windows), whereas the intensity increased as the cells migrated into the collection channel (white rectangular windows). EpCAM had a similar oscillation pattern, but the mean intensity of EpCAM was lower than that of Vimentin (FIG. 3C, right). It was hypothesized that cells in the collection channel tended to relax and shrink into sphere-like structures, so the value of intensity per area increased. The assumption was confirmed by the intensity ratio of vimentin and EpCAM (FIG. 8), which shows that cells in the migration channels had a relatively higher “Vimentin to EpCAM” ratio as compared with the cells in the collection channels. Besides, ratios were larger for cells with longer migration distance (Channel #6-9), indicating that highly migratory cells had a higher vimentin expression level and lower EpCAM expression level and thus could be used as an indicator to predict migratory behaviors of cancer cells.

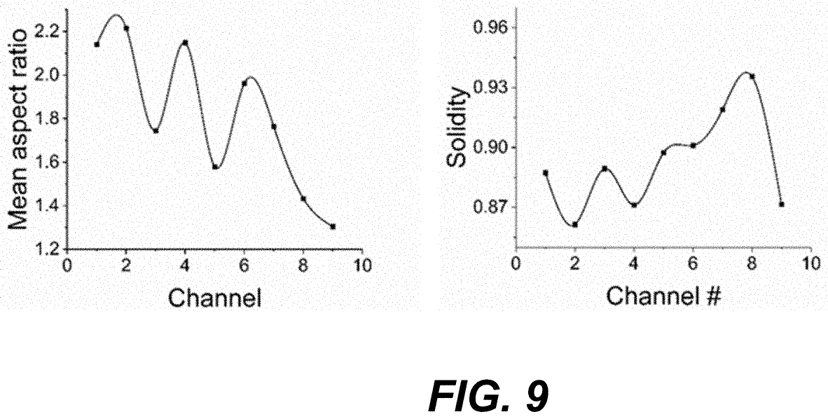

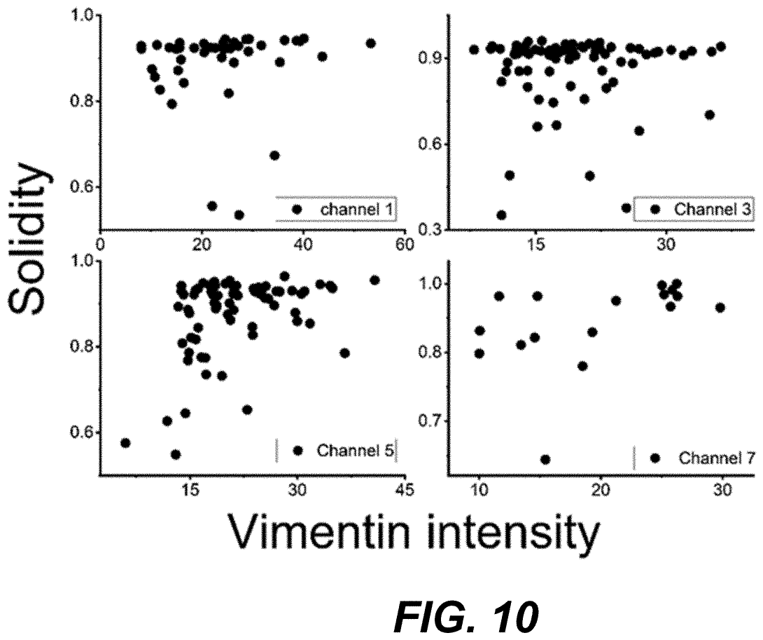

Moreover, cell migration patterns could also be analyzed with shape description parameters including aspect ratio, which was used to indicate the intrinsic ability to elongate, and solidity, which was used to represent the cell's ability to form protrusions. Cells in the migration channels typically have higher aspect ratio and lower solidity (FIG. 3D and FIG. 9) indicating a higher ability to deform, whereas lower aspect ratio and higher solidity happens for cells in the collection channels. The Non-migratory cells, cells in the collection channel, typically had a relatively lower vimentin expression level and lower aspect ratio. Migratory cells, cells in the migration channel (next to the compared collection channel), usually had a higher vimentin expression level, were easier to deform and send protrusions, thus had a higher aspect ratio. Given these findings, it is possible to correlate the aspect ratio to vimentin expression level. As shown in FIG. 3E, there was no strong linear relationship (R²=0.001) between the vimentin intensity and aspect ratio when data for all of the cells were taken together. Intriguingly, good linear regressing fit happened when cells from induvial collection channels were plotted (#1, #3, #5 and #7) as well as the migration channel (#2, #4, #6 and #8) with a R² value of 0.94, 0.95, 0.96 and 0.95, and 0.97, 0.94, 0.95 and 0.99, respectively. There is no strong relationship between vimentin intensity and solidity (FIG. 10). Based on these findings, it was concluded that vimentin and aspect ratio could be parameters to illustrate the migratory behaviors of cancer cells in MChip.

Comparison of Migratory Behaviors of Different Cancer Cell Lines

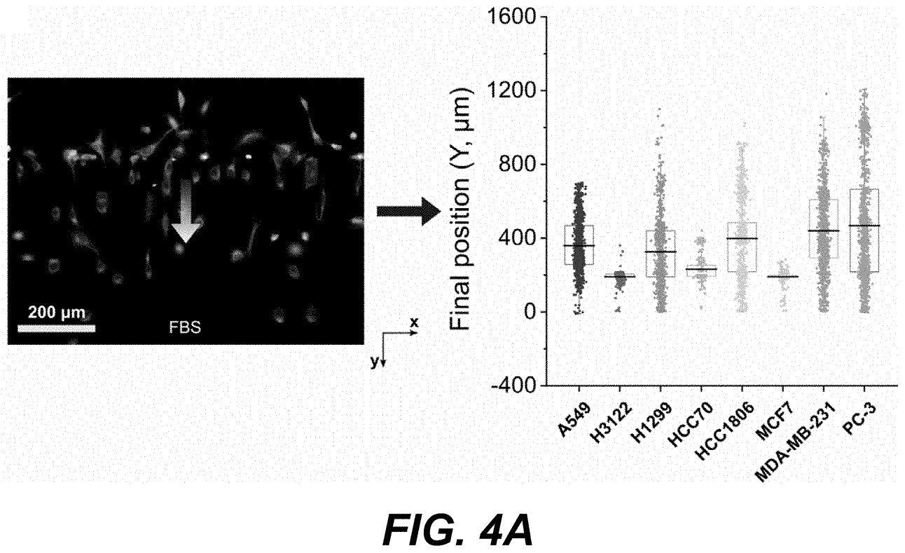

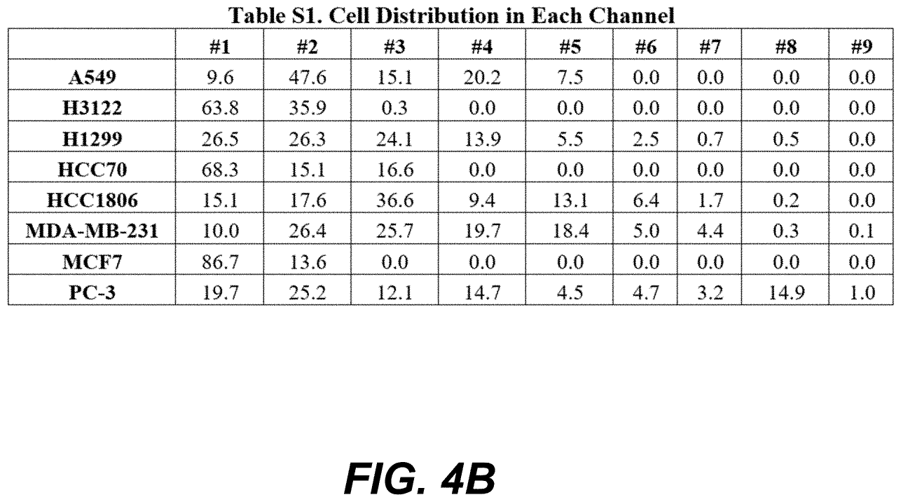

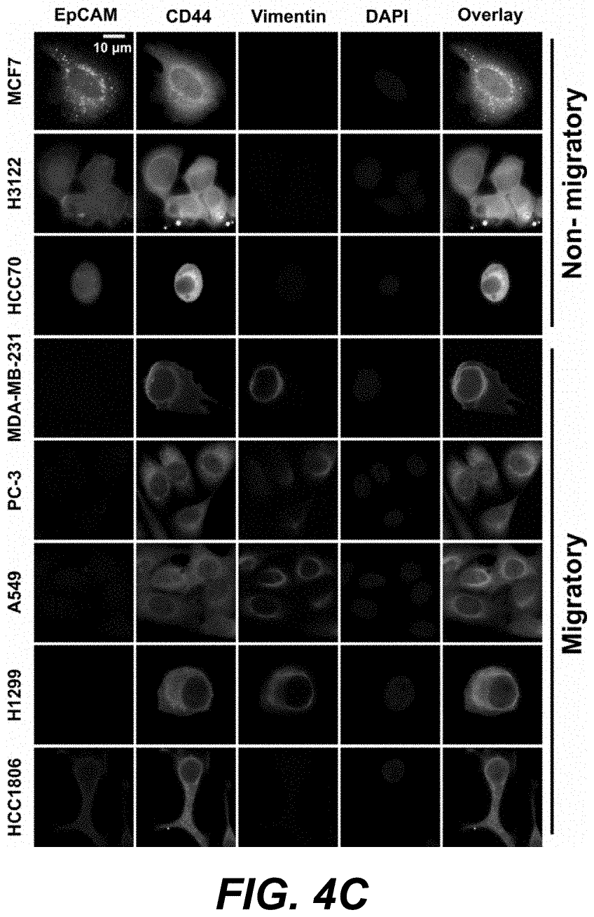

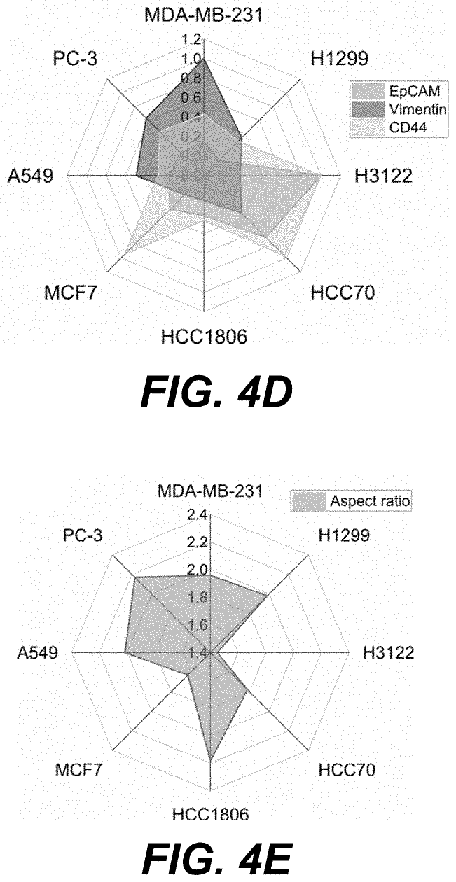

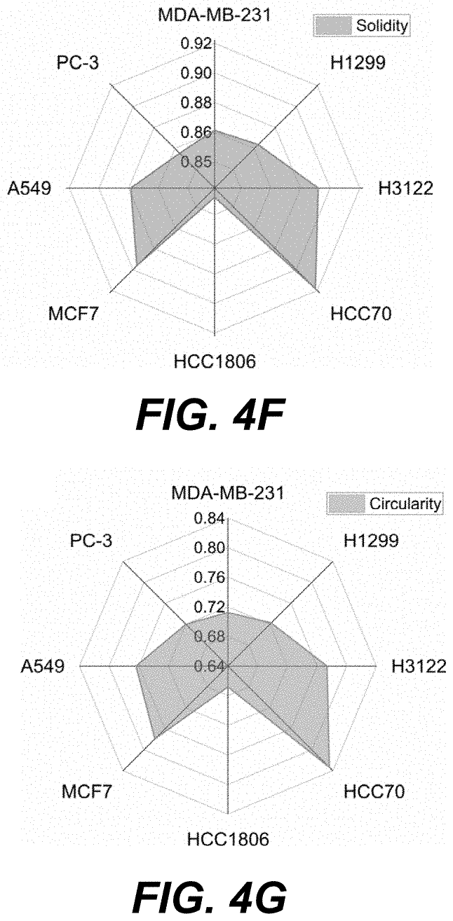

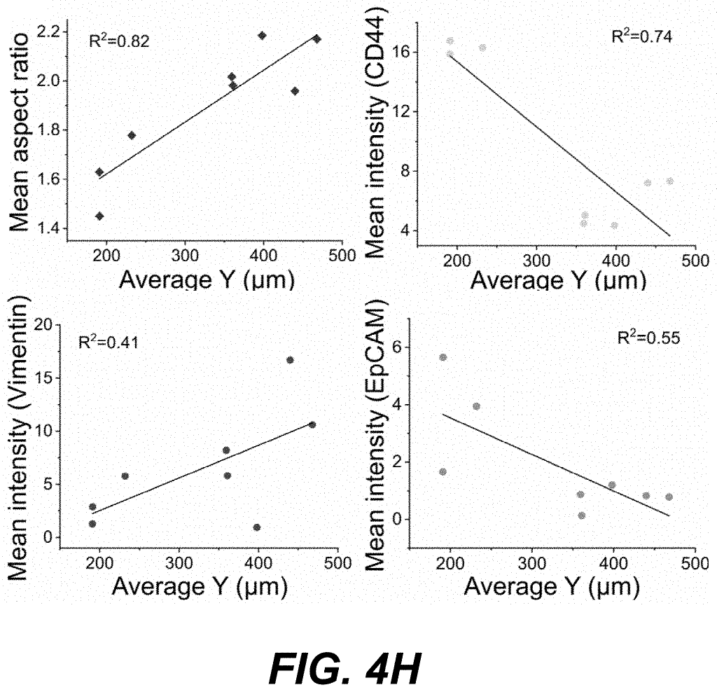

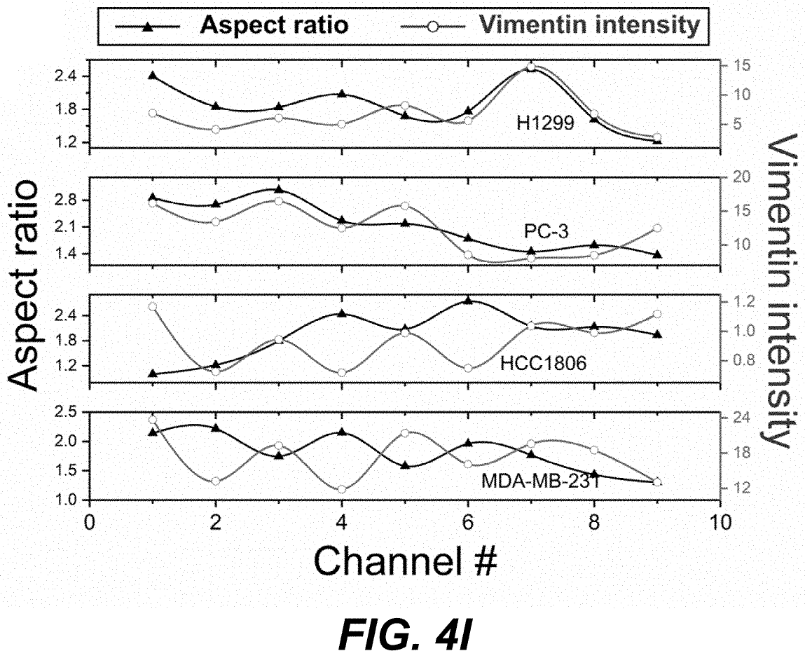

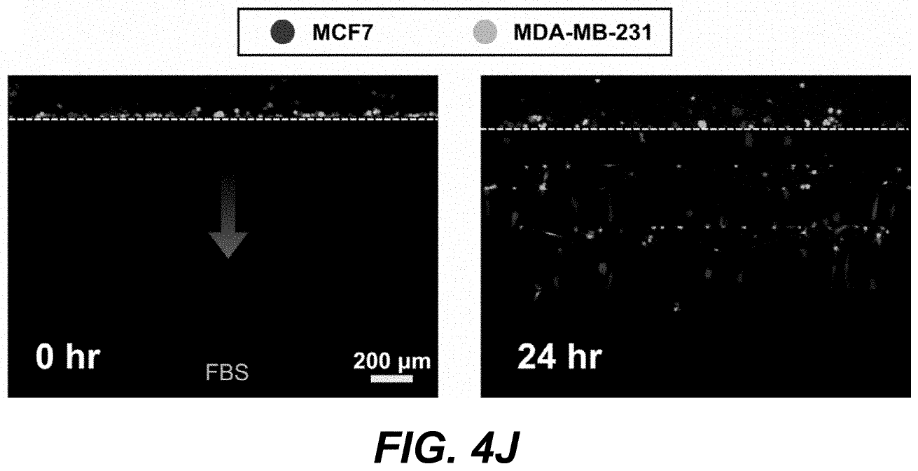

To further validate aspect ratio and vimentin expression level and explore the potential parameters in the contribution to migratory behaviors, 3 non-small cell lung cancer (NSCLC) cell lines, 4 breast cancer cell lines, and 1 prostate cancer cell line were tested. Typical migration assays were performed under optimized conditions discussed above (chemo-attractant: 10% FBS, migration time: 24 hours), following by fixing and immunofluorescence staining of cells on MChip (FIG. 4A, left). The final positions, determined by the nucleus position, of 8 cancer cell lines were summarized in FIG. 4A (right). The average final position of individual cells and migration distribution varied between different cell lines. A549, H1299, HCC1806, MDA-MB-231 and PC-3 were relatively migratory cell lines with average final positions larger than 300 μm. H3122, HCC70 and MCF7, on the other hand, were non-migratory cell lines with average final positions smaller than 300 μm. The table in FIG. 4B shows the percentage of cells in each channel. Non-migratory cell line, MCF7, had 86.7% of cells stayed in the cell loading channel (#1) and 13.6% of cells in the migration channel (#2), but the final positions for most of the MCF7 cells in the migration channel were smaller than 250 μm (moved 1/3 of the migration channel). This was also true for breast cancer cell line H3122. HCC70 was a relative non-migratory cell line with 68.3% cells stick to the cell loading channel, whereas 16.6% of cells could still deform and migrate into the first collection channel (#3). PC-3 had the highest average final position and broadest distribution with 12.1%, 4.5% and 3.2% in collection channels (#3, #5 and #7, respectively), and 25.2, 14.7%, 4.7% and 14.9% in the migration channels (#2, #4, #6 and #8, respectively). MDA-MB-231 was the only cell line with more than 90% of cells left the cell loading channel.

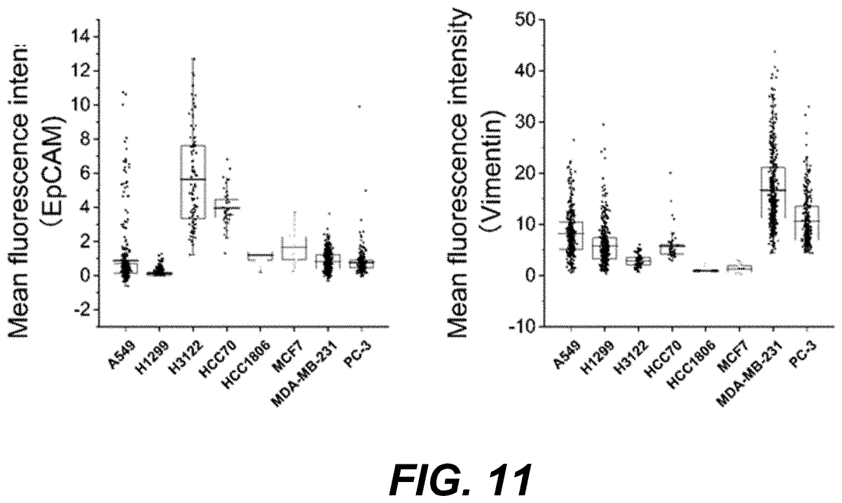

In light of findings from MDA-MB-231 migration assays, it was decided to test the relationship between EMT marker expression and migratory behaviors of different cancer cell lines. Non-migratory cells lines, as shown in FIGS. 4C-4D and FIG. 11, had higher EpCAM intensity and lower or even no vimentin intensity. On the contrary, migratory cell lines, including MDA-MB-231, PC-3, A549, and H1299 had lower EpCAM intensity and higher vimentin intensity. The “Low EpCAM and High Vimentin” works well to predict migratory cell lines except for HCC1806, which is invasive triple-negative breast cancer (TNBC) cell line and had reduced vimentin intensity and increased EpCAM intensity in the MChip assay. TNBC is cancer that doesn't have estrogen or progesterone receptors and has less production of a protein called HER2, and it usually triggers higher tumor mitotic index²⁸ and a worse prognosis in patient.²⁹⁻³⁰ Despite many successful treatments of TNBCs, there is little knowledge about the specific characteristic and parameters of this cancer that could predict its migratory properties and tumor recurrence. To comprehensively illustrate the migratory behavior of different cancer cell lines, we add a cancer stem marker, CD44, to our prediction model. As shown in the IF staining images, CD44 had a lower expression level in migratory cells, including TNBC cell line HCC1806, and was highly expressed in non-migratory cells. Although the majority of in vitro studies show that CD44 could support cancer progression, invasion, and migration³¹, numerous reports show that CD44 could inhibit cancer growth and invasion due to the interactions between CD44 and cues from the microenvironment³². We hypothesized that it was the CD44′s response to collagen type I in the microenvironment that contributed to the CD44′s loss in the migratory cell lines and thus bestowed cells with high migratory properties. This could be confirmed by the channel coating experiment (FIG. 7), in which optimal coating material for MDA-MB-231 migration assay was collagen type I instead of Poly-L-Lysine.

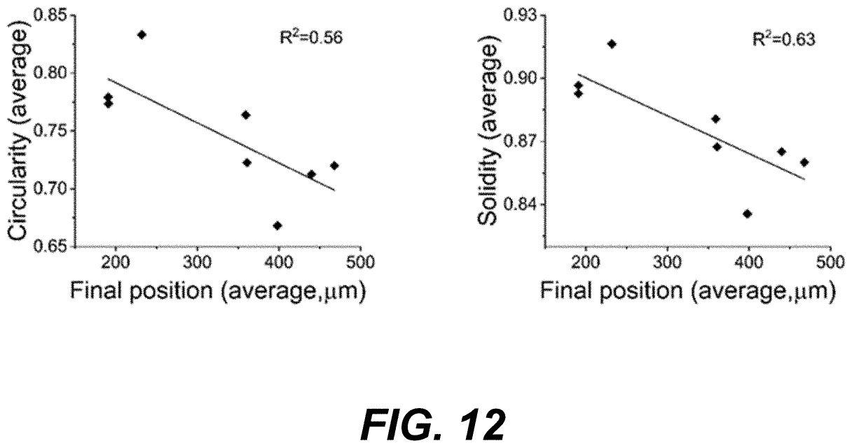

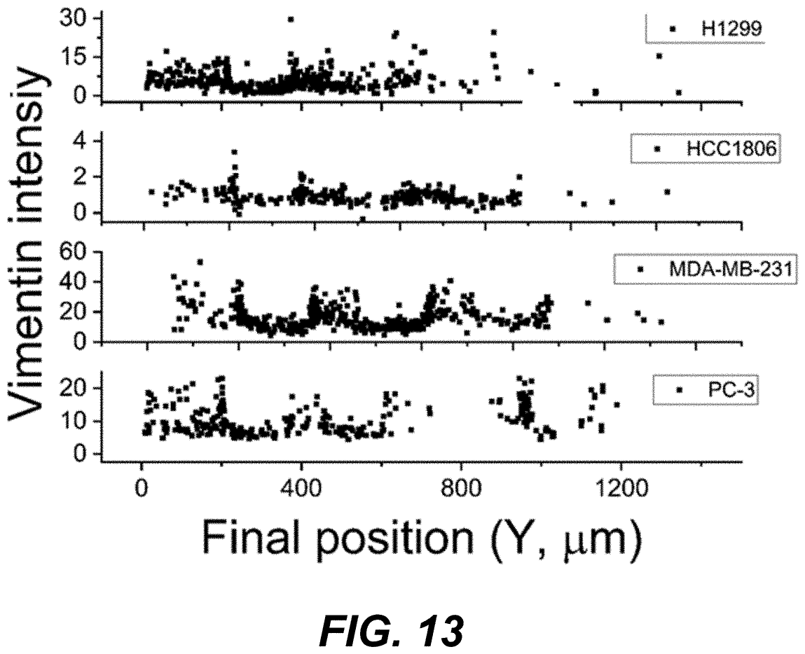

The migration patterns of different cancer cell lines were also represented with shape descriptions, including aspect ratio (FIG. 4E), solidity (FIG. 4F) and circularity (FIG. 4G). On one hand, migratory cell lines, with an average final position larger than 300 μm, had a higher aspect ratio, lower solidity, and circularity due to their capability to deform in the confined space. One the other hand, non-migratory cell lines, with an average final position smaller than 300 μm, had a lower aspect ratio, higher solidity, and circularity indicating their reduced ability to invade. Linear regression analysis (FIG. 4H, and FIG. 12) showed a positive correlation between average final position and mean aspect ratio (R²=0.82, P=0.002), whilst a negative correlation between average final position and CD44 intensity (R²=0.74, P=0.006). In view of the findings, it was concluded that CD44 could be a better single parameter in the prediction of cancer migratory behaviors than EpCAM and vimentin, while aspect ratio had a stronger correlation with the final position as compared with solidity and circularity. However, multiple linear regression shows that the combination of vimentin and aspect ratio showed the strongest correlation with the average final position (R²=0.94, P=0.001). Although the combination of mean vimentin intensity and aspect ratio were good parameter in illustration of cell migration, behaviors of individual cells in the migration channels and collection channels varies among different cell lines. FIG. 4I and FIG. 13 show the vimentin intensities and aspect ratio in each channel for migratory cell lines, H1299, PC-3, HCC1806, and MDA-MB-231. Vimentin intensity oscillations happened with peak values in the collection channels, but the oscillations patterns of aspect ratio were different from each other. This could be explained by different migration mechanisms and interaction strength between collagen and cell surface markers, for example, CD44.

To test the ability of MChip to identify and isolate migratory cells from non-migratory cells, invasive breast cancer cell line, MDA-MB-231, were mixed (at a ratio of 1:1) with non-invasive breast cancer cell line, MCF7, pre-labeled with two different fluorophores and loaded a total of 5000 cells into the microfluidic platform (FIG. 4J). The assay was performed under optimized conditions (0.1 μL/min, 10% FBS). Through the percentage of MDA-MB-231 cancer cells leaving loading channel (#1) is proportional to the time, 24 hours was enough for more than 90% of MDA-MB-231 to move into the migration channels and collected with about 100% purity.

Cell Viability and Proliferation Study

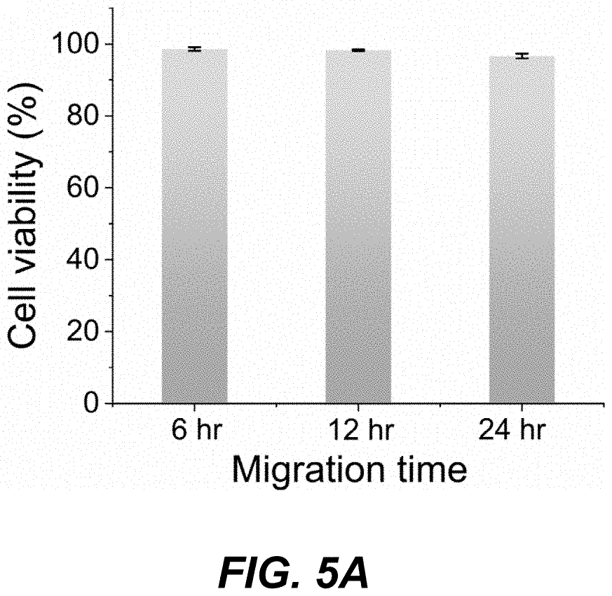

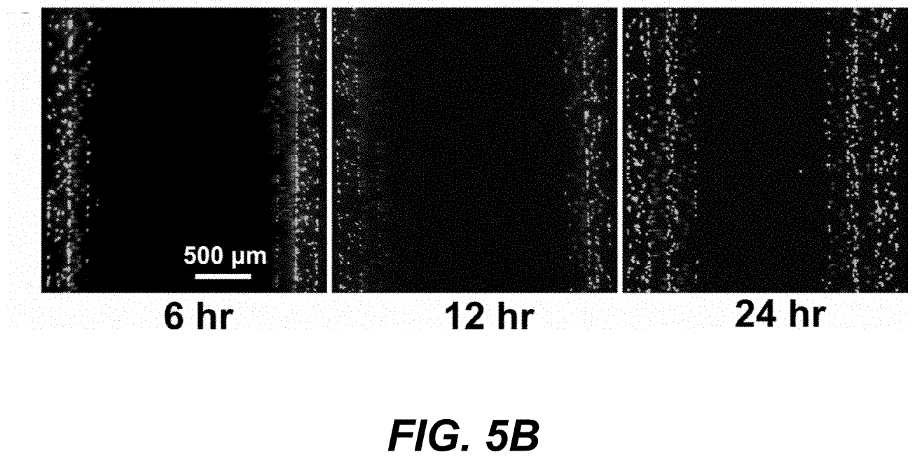

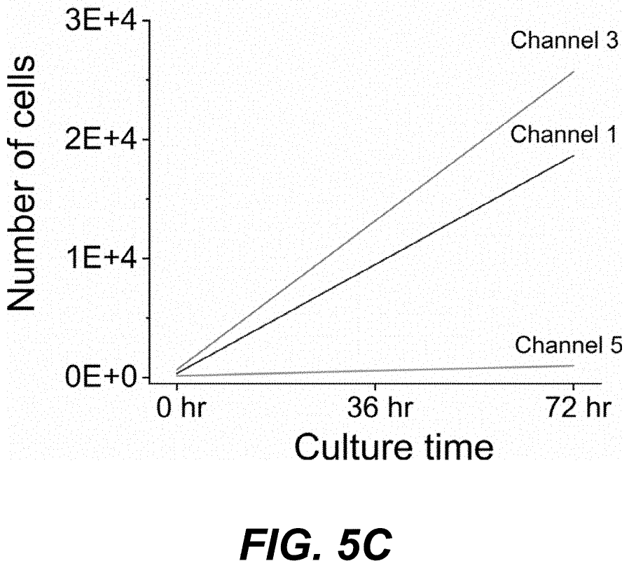

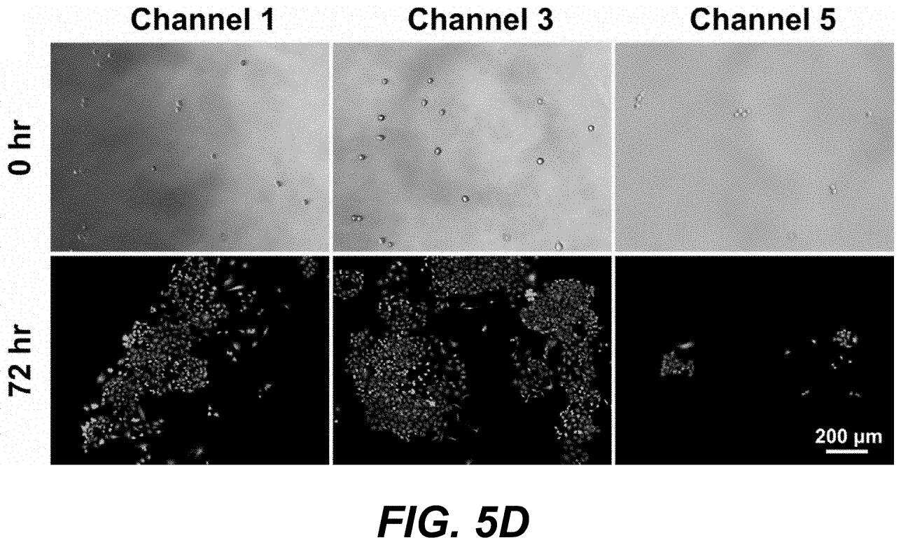

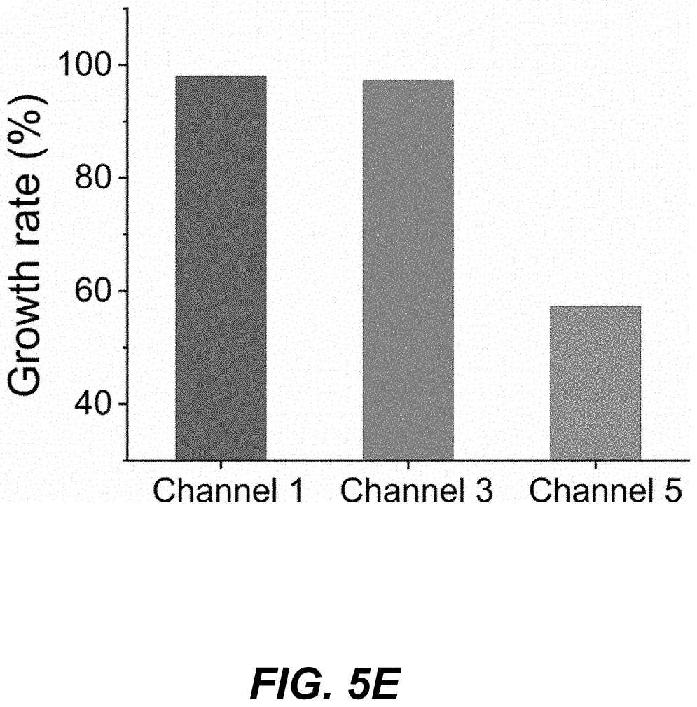

The MChip migration process had little impact on cell viability and intactness for three reasons: (1) the area of migration channel's cross-section was 150 μm², which was larger than the 20 μm²'s threshold to cause nuclear envelope rupture and DNA damage.^{25, 33} (2) the whole system was placed in the incubator (37° C., 5% CO2) that is suitable to maintain cell viability. (3) a continuous flow was applied to refresh the medium and remove waste. The short-term cell viability on the migration chip and long-term cell proliferation of cancer cells following the retrieving process after migration assay was examined. As shown in FIG. 5A, the cell viability of H1299 NSCLC cells after 6 hours, 12 hours and 24 hours' migration under optimized conditions (0.1 μL/min, 10% FBS) was determined to be 98.63%±0.47%, 98.33±0.21% and 96.67±0.63%, respectively, indicating a negligible decrease in cell viability on the MChip within 24 hours. Representative fluorescence images of cells are shown in FIG. 5B. Whether retrieved cancer cells from different collection channels continued to proliferate normally was also investigated. FIG. 5D shows the images of collected H1299 NSCLC cells on the third day. The fluorescence image in FIG. 5D also confirmed that cells were viable after 3 days' culture. Cells from collection channel #1 and #2 were able to proliferate to confluence and maintain the morphology after the MChip process, whereas cells from collection channel #3 couldn't proliferate to confluence. The variable number of cells (FIG. 5C) and growth rates (FIG. 5E) in different channels were due to the different proliferation ability: (1) cell density was too low. (2) long migration distance will reduce the proliferation ability, due to the potential DNA damage or molecular signature alteration.

Combination of iFCS and MChip to Achieve Highly Purified CTCs.

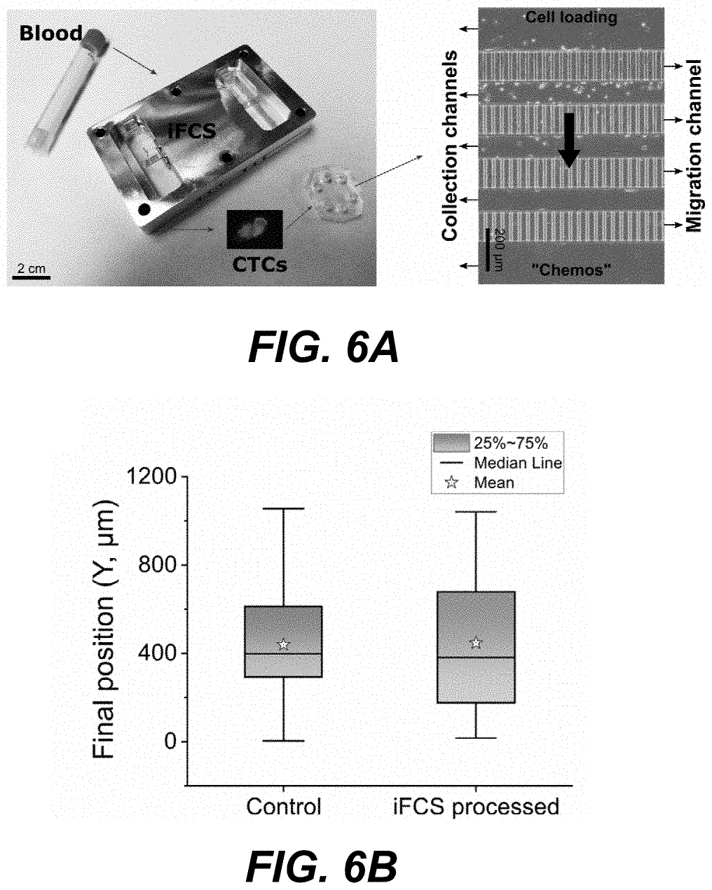

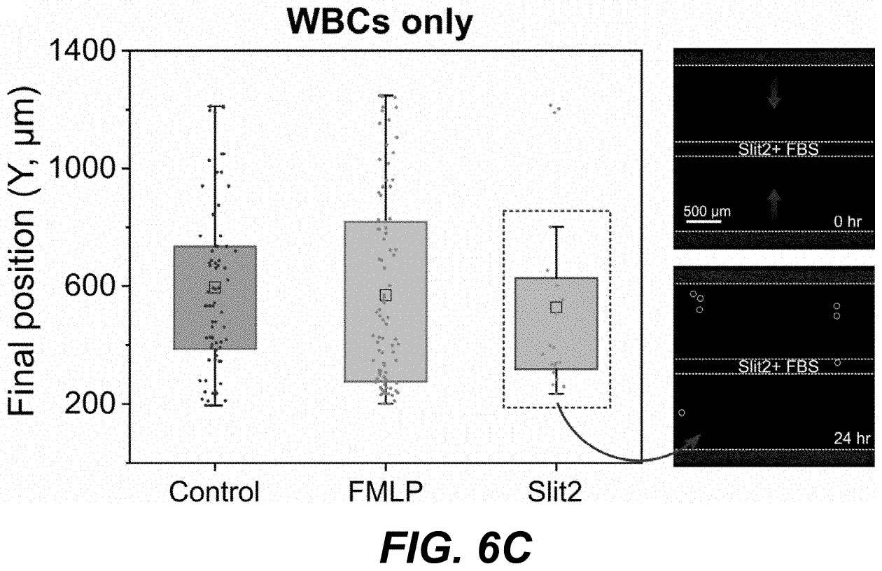

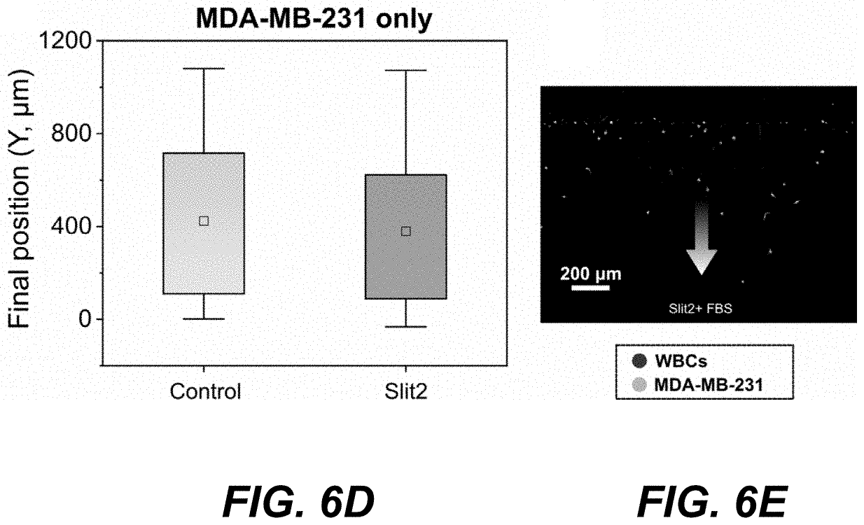

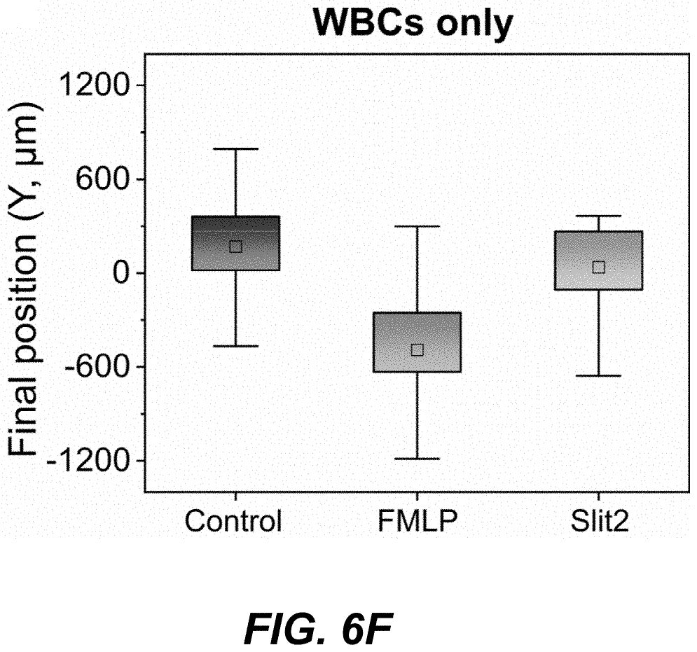

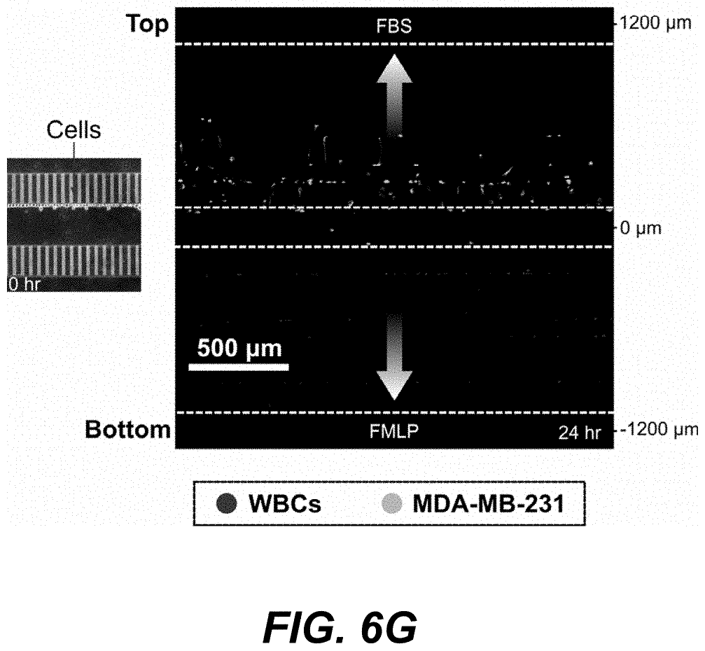

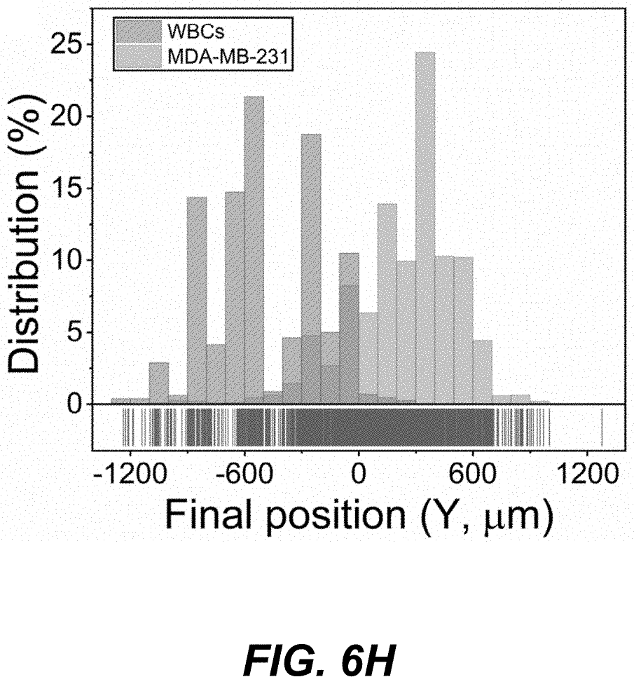

Through previous work¹⁰ (Zhao, et al., incorporated by reference above) a microfluidic technique (iFCS) was developed to enrich/isolate circulating tumor cells from cancer patient blood, with a 99.1% recovery rate and low WBC contamination (˜500 WBCs for every one milliliter blood processed). However, there were still about 5000 WBCs after processing 10 mL blood, while the CTCs were extremely rare (10˜1000), which could increase the downstream processing time and cost and introduce high background noise. FIG. 6A shows the working flow of the purification, the combination of iFCS and MChip provide a stage to further remove WBCs using varied migratory behaviors under optimized chemo-attractant loading and concentration and give us super-purified CTC sample. iFCS had little effect on cell viability and proliferation ability, but the effect on migratory properties was not studied. To test the iFCS processing effect on the migratory behaviors of cancer cells, MDA-MB-231 cancer cells were spiked into healthy human blood, retrieved cancer cells with iFCS and loaded into MChip to perform migration assay. The average final positions of MDA-MB-231 cancer cells had no significant change (P=0.46) after iFCS processing, as shown in FIG. 6B.

To preserve the migration properties of cancer cells while inhibiting the movement of WBCs, different types and combinations of chemo-attractant or chemo-repellent were tested, such as FBS, FMLP, and slit2. It was shown that only 0.4% of WBCs were left the cell loading channel (#1) when the middle channel was loaded with 10% FBS (cancer cell attractant) and 5 μg/mL slit2 (WBC repellant) (FIG. 6C). As shown in FIGS. 6D-6E, 5 μg/mL slit2, the neutrophil inhibitor could slightly compromise the migratory behavior of MDA-MB-231 cancer cells (P=0.004), but there was still 85% of cancer cells squeezed into the migration channel. However, non-migratory cells in the loading channel were contaminated with 99.6% of loaded WBCs.

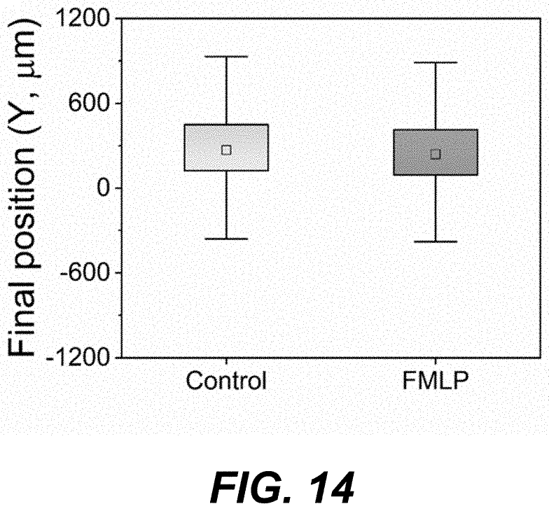

To retrieve the cancer cells of heterogeneously migratory properties with low WBCs contamination, the cell loading channel and “chemos” loading channel (middle, #9) were swapped: cells was loaded to the top part of the middle channel (#9) while the serum was loaded into side channels (top/bottom, #1). FM LP, the neutrophil chemo-attractant, was loaded into the bottom side channel while slit2 was filled in the top side channel. FMLP could attract the majority of WBCs to the bottom part of MChip (FIG. 6F) without a negative effect on the MDA-MB-231 migration (FIG. 14). Furthermore, a mixture of MDA-MB-231 cancer cells and WBCs (at a ratio of 1:1) was loaded into the top part of the middle (main) channel (FIG. 6G, left). Cell culture medium with 10% FBS, 0% FBS and 0% FBS +FMLP was injected (0.1 μL/min) into top side channel, middle channel and bottom side channel, respectively. MDA-MB-231 cancer cells moved to the top part while the WBCs migrated to the bottom part (FIG. 6G, right). The cells were quantified at different final positions: about 0.5% of WBCs and 76.3% of MDA-MB-231 cells migrated to the top part; 11.2% of WBCs and 14.5% of MDA-MB-231 cells stayed in the middle loading channel; 88.3% of WBCs and 9.2% of MDA-MB-231 cells moved to the bottom part (FIG. 6H). This loading method could isolate non-migratory cancer cells with the lowest WBC contamination. Since MChip can retrieve cancer cells from each migration channel and collection channel, the purity will vary from channel to channel.

Conclusion

The present example demonstrates the design and function of a new microfluidic device (MChip) to study the migratory properties of target cells, such as heterogeneous cancer cells. In embodiments, MChip applies chemo-attractant forces that can attract migratory cells while non-migratory cells stay near the starting point. An analytical model was also provided to predict the flow profile and concentration gradient in the microfluidic channels to guide the optimization processes of cell loading and determine the appropriate operating parameters. The optimized design could simultaneously study 5000 cells with little effect on cell viability and proliferation ability and could classify cancer cells into as many as 9 subtypes with the help of collection channels. To illustrate the migratory behaviors of different cancer cell lines, shape descriptions and biological markers were used to predict the average final position: (1) aspect ratio of cells had a positive correlation with cell final position; (2) cancer stem marker (CD44) had a negative correlation with migration distance; (3) combination of aspect ratio and vimentin provided a strong predictive value in the illustration of cell migration. Moreover, the combination of MChip and iFCS can provide a super-purified CTCs sample using chemo-attractant and chemo-repellent compounds loaded into different channels, resulting in purification of migratory cancer cells very low WBCs contamination (e.g., 2 cells) after processing 1 mL of blood.

### REFERENCES

1. Siegel, R. L.; Miller, K. D.; Jemal, A., Cancer statistics, 2020. CA Cancer J Clin 2020, 70 (1), 7-30.
2. Steeg, P. S., Targeting metastasis. Nat Rev Cancer 2016, 16 (4), 201-18.
3. Poudineh, M.; Sargent, E. H.; Pantel, K.; Kelley, S. 0., Profiling circulating tumour cells and other biomarkers of invasive cancers. Nat Biomed Eng 2018, 2 (2), 72-84.
4. Chaffer, C. L.; Weinberg, R. A., A perspective on cancer cell metastasis. Science 2011, 331 (6024), 1559-64.
5. Zhao, W.; Cheng, R.; Jenkins, B. D.; Zhu, T.; Okonkwo, N. E.; Jones, C. E.; Davis, M. B.; Kavuri, S. K.; Hao, Z.; Schroeder, C.; Mao, L., Label-free ferrohydrodynamic cell separation of circulating tumor cells. Lab Chip 2017, 17 (18), 3097-3111.
6. Weitz, J.; Kienle, P.; Lacroix, J.; Willeke, F.; Benner, A.; Lehnert, T.; Herfarth, C.; von Knebel Doeberitz, M., Dissemination of tumor cells in patients undergoing surgery for colorectal cancer. Clin Cancer Res 1998, 4 (2), 343-8.
7. Qin, X.; Park, S.; Duffy, S. P.; Matthews, K.; Ang, R. R.; Todenhofer, T.; Abdi, H.; Azad, A.; Bazov, J.; Chi, K. N.; Black, P. C.; Ma, H., Size and deformability based separation of circulating tumor cells from castrate resistant prostate cancer patients using resettable cell traps. Lab Chip 2015, 15 (10), 2278-86.
8. Riethdorf, S.; Fritsche, H.; Muller, V.; Rau, T.; Schindlbeck, C.; Rack, B.; Janni, W.; Coith, C.; Beck, K.; Janicke, F.; Jackson, S.; Gornet, T.; Cristofanilli, M.; Pantel, K., Detection of circulating tumor cells in peripheral blood of patients with metastatic breast cancer: a validation study of the CellSearch system. Clin Cancer Res 2007, 13 (3), 920-8.
9. Talasaz, A. H.; Powell, A. A.; Huber, D. E.; Berbee, J. G.; Roh, K.-H.; Yu, W.; Xiao, W.; Davis, M. M.; Pease, R. F.; Mindrinos, M. N., Isolating highly enriched populations of circulating epithelial cells and other rare cells from blood using a magnetic sweeper device. Proceedings of the National Academy of Sciences 2009, 106 (10), 3970-3975.
10. Zhao, W.; Liu, Y.; Jenkins, B. D.; Cheng, R.; Harris, B. N.; Zhang, W.; Xie, J.; Murrow, J. R.; Hodgson, J.; Egan, M., Tumor antigen-independent and cell size variation-inclusive enrichment of viable circulating tumor cells. Lab on a Chip 2019, 19 (10), 1860-1876.
11. Rodriguez, L. G.; Wu, X.; Guan, J.-L., Wound-healing assay. In Cell Migration, Springer: 2005; pp 23-29.
12. Chen, H.-C., Boyden chamber assay. In Cell migration, Springer: 2005; pp 15-22.
13. Chen, Y.-C.; Humphries, B.; Brien, R.; Gibbons, A. E.; Chen, Y.-T.; Qyli, T.; Haley, H. R.; Pirone, M. E.; Chiang, B.; Xiao, A., Functional isolation of tumor-initiating cells using microfluidic-based migration identifies phosphatidylserine decarboxylase as a key regulator. Scientific reports 2018, 8 (1), 1-13.
14. Yankaskas, C. L.; Thompson, K. N.; Paul, C. D.; Vitolo, M. I.; Mistriotis, P.; Mahendra, A.; Bajpai, V. K.; Shea, D. J.; Manto, K. M.; Chai, A. C.; Varadarajan, N.; Kontrogianni-Konstantopoulos, A.; Martin, S. S.; Konstantopoulos, K., A microfluidic assay for the quantification of the metastatic propensity of breast cancer specimens. Nat Biomed Eng 2019, 3 (6), 452-465.
15. Chen, Y. C.; Allen, S. G.; Ingram, P. N.; Buckanovich, R.; Merajver, S. D.; Yoon, E., Single-cell Migration Chip for Chemotaxis-based Microfluidic Selection of Heterogeneous Cell Populations. Sci Rep 2015, 5, 9980.
16. Chen, M. B.; Whisler, J. A.; Frose, J.; Yu, C.; Shin, Y.; Kamm, R. D., On-chip human microvasculature assay for visualization and quantification of tumor cell extravasation dynamics. Nat Protoc 2017, 12 (5), 865-880.
17. Frick, C.; Dettinger, P.; Renkawitz, J.; Jauch, A.; Berger, C. T.; Recher, M.; Schroeder, T.; Mehling, M., Nano-scale microfluidics to study 3D chemotaxis at the single cell level. PloS one 2018, 13 (6).
18. Jeon, J. S.; Bersini, S.; Gilardi, M.; Dubini, G.; Charest, J. L.; Moretti, M.; Kamm, R. D., Human 3D vascularized organotypic microfluidic assays to study breast cancer cell extravasation. Proceedings of the National Academy of Sciences 2015, 112 (1), 214-219.
19. Lambert, A. W.; Pattabiraman, D. R.; Weinberg, R. A., Emerging Biological Principles of Metastasis. Cell 2017, 168 (4), 670-691.
20. Joyce, J. A.; Pollard, J. W., Microenvironmental regulation of metastasis. Nat Rev Cancer 2009, 9 (4), 239-52.
21. Chen, Y. C.; Humphries, B.; Brien, R.; Gibbons, A. E.; Chen, Y. T.; Qyli, T.; Haley, H. R.; Pirone, M. E.; Chiang, B.; Xiao, A.; Cheng, Y. H.; Luan, Y.; Zhang, Z.; Cong, J.; Luker, K. E.; Luker, G. D.; Yoon, E., Functional Isolation of Tumor-Initiating Cells using Microfluidic-Based Migration Identifies Phosphatidylserine Decarboxylase as a Key Regulator. Sci Rep 2018, 8 (1), 244.
22. Trzpis, M.; McLaughlin, P. M.; de Leij, L. M.; Harmsen, M. C., Epithelial cell adhesion molecule: more than a carcinoma marker and adhesion molecule. Am J Pathol 2007, 171 (2), 386-95.
23. Cui, X.; Guo, W.; Sun, Y.; Sun, B.; Hu, S.; Sun, D.; Lam, R. H. W., A microfluidic device for isolation and characterization of transendothelial migrating cancer cells. Biomicrofluidics 2017, 11 (1), 014105.
24. Satelli, A.; Li, S., Vimentin in cancer and its potential as a molecular target for cancer therapy. Cell Mol Life Sci 2011, 68 (18), 3033-46.
25. Paul, C. D.; Mistriotis, P.; Konstantopoulos, K., Cancer cell motility: lessons from migration in confined spaces. Nat Rev Cancer 2017, 17 (2), 131-140.
26. Schoumacher, M.; Goldman, R. D.; Louvard, D.; Vignjevic, D. M., Actin, microtubules, and vimentin intermediate filaments cooperate for elongation of invadopodia. J Cell Biol 2010, 189 (3), 541-56.
27. Paccione, R. J.; Miyazaki, H.; Patel, V.; Waseem, A.; Gutkind, J. S.; Zehner, Z. E.; Yeudall, W. A., Keratin down-regulation in vimentin-positive cancer cells is reversible by vimentin RNA interference, which inhibits growth and motility. Mol Cancer Ther 2008, 7 (9), 2894-903.
28. Umemura, S.; Takekoshi, S.; Suzuki, Y.; Saitoh, Y.; Tokuda, Y.; Osamura, R. Y., Estrogen receptor-negative and human epidermal growth factor receptor 2-negative breast cancer tissue have the highest Ki-67 labeling index and EGFR expression: gene amplification does not contribute to EGFR expression. Oncology reports 2005, 14 (2), 337-343.
29. Jacquemier, J.; Ginestier, C.; Rougemont, J.; Bardou, V. J.; Charafe-Jauffret, E.; Geneix, J.; Adelaide, J.; Koki, A.; Houvenaeghel, G.; Hassoun, J.; Maraninchi, D.; Viens, P.; Birnbaum, D.; Bertucci, F., Protein expression profiling identifies subclasses of breast cancer and predicts prognosis. Cancer Res 2005, 65 (3), 767-79.
30. Volk-Draper, L. D.; Rajput, S.; Hall, K. L.; Wilber, A.; Ran, S., Novel model for basaloid triple-negative breast cancer: behavior in vivo and response to therapy. Neoplasia 2012, 14 (10), 926-42.
31. Louderbough, J. M.; Schroeder, J. A., Understanding the dual nature of CD44 in breast cancer progression. Mol Cancer Res 2011, 9 (12), 1573-86.
32. Naor, D.; Nedvetzki, S.; Golan, I.; Melnik, L.; Faitelson, Y., CD44 in cancer. Critical reviews in clinical laboratory sciences 2002, 39 (6), 527-579.
33. Denais, C. M.; Gilbert, R. M.; lsermann, P.; McGregor, A. L.; to Lindert, M.; Weigelin, B.; Davidson, P. M.; Friedl, P.; Wolf, K.; Lammerding, J., Nuclear envelope rupture and repair during cancer cell migration. Science 2016, 352 (6283), 353-8.

We claim:

1. A device for enrichment, separation, and subtyping of circulating target cells in a biological sample fluid, the device comprising:
a microfluidic chip substrate having a plurality of microfluidic channels formed thereon, the microfluidic channels comprising:
a main channel having a first end and a second end, a first inlet at the first end wherein the inlet is configured to flow a first fluid into the main channel, and a first outlet at the second end configured to collect contents of the first fluid that exit the main channel;
a first outer channel oriented substantially parallel to and on a first side of the main channel and having a first end and a second end, a second inlet at the first end of the first outer channel wherein the second inlet is configured to flow a second fluid into the first outer channel, and a second outlet at the second end of the first outer channel;
a first-side plurality of microchannels connecting the main channel to the first outer channel and oriented substantially perpendicular to the main channel and first outer channel, wherein the first-side plurality of microchannels are in fluidic communication with both the main channel and the first outer channel; and
one or more first-side collection channels located in between and oriented substantially parallel to both the main channel and first outer channel and in fluidic communication with the first-side plurality of microchannels, the one or more first-side collection channels each having a first and second end and each first-side collection channel being in fluidic communication with a first flushing port at the first end and each having an individual collection outlet at the second end.

2. The device of claim 1, configured such that target cells in the sample fluid migrate toward or away from the channel flowing the chemo-modulatory fluid in response to the chemogradient, wherein non-target cells in the sample migrate toward or away from the channel flowing the chemo-modulatory fluid in response to the chemogradient, or both, and wherein the movement of the target cells is distinguishable from the movement of the non-target cells.

3. The device of claim 2, wherein each of the main channel and first outer channel independently has a width of about 50 μm to 1000 μm, a height of about 30 μm to 150 μm, and a length of about 10 mm to 60 mm.

4. The device of claim 1, wherein each of the one or more first-side collection channels has a width of about 50 μm to 200 μm and a height of about 30 μm to 150 μm.

5. The device of claim 4, wherein each of the first-side plurality of microchannels has a width of about 8 μm to 40 μm, a height of about 3 μm to 7 μm, and a length of about 150 μm.

6. The device of claim 1, further comprising:
a second outer channel on a second side of the main channel opposite the first outer channel, the second outer channel oriented substantially parallel to the main channel and first outer channel and having a first end and a second end, a third inlet at the first end of the second outer channel wherein the third inlet is configured to flow a third fluid into the second outer channel, and a third outlet at the second end of the second outer channel;
a second-side plurality of microchannels connecting the main channel to the second outer channel and oriented substantially perpendicular to the main channel and second outer channel, wherein the microchannels are in fluidic communication with the main channel and the second outer channel; and
one or more second-side collection channels located in between and oriented substantially parallel to both the main channel and second outer channel and in fluidic communication with the second-side plurality of microchannels, the one or more second-side collection channels each having a first and second end and each second-side collection channel being in fluidic communication with a second flushing port at the first end and each having an individual collection outlet at the second end.

7. The device of claim 3, wherein the second outer channel, second-side plurality of micro-channels, and one or more second-side collection channels have a configuration that substantially mirrors a configuration of the first outer channel, first-side plurality of micro-channels and one or more first-side collection channels.

8. A kit comprising: the device of claim 1, at least one chemo-modulatory fluid or instructions for preparing at least one chemo-modulatory fluid, and instructions for use of the device of claim 1 and the at least one chemo-modulatory fluid to separate target cells from a biological sample fluid.

9. The kit of claim 8, wherein the biological sample fluid is a sample from a subject in which target cells have been partially enriched.

10. The kit of claim 8, wherein the target cells are CTCs and wherein the chemo-modulatory fluid comprises a chemo-attractant that attracts circulating tumor cells (CTCs), a chemo-repellent that repels white blood cells (WBCs), or a combination of both.

11. A kit comprising: the device of claim 6, at least one chemo-modulatory fluid or instructions for preparing at least one chemo-modulatory fluid, and instructions for use of the device of claim 6 and the at least one chemo-modulatory fluid to separate target cells from a biological sample.

12. The kit of claim 11, wherein the target cells are CTCs and wherein the chemo-modulatory fluid comprises a chemo-attractant that attracts circulating tumor cells (CTCs), a chemo-repellent that repels white blood cells (WBCs), or a combination of both.

13. A method for separating target cells in a biological sample, the method comprising:
in the device of claim 1, introducing a chemo-modulatory fluid in the first inlet of the main channel or the second inlet of the first outer channel, such that the chemo-modulatory fluid flows in the device and establishes a chemogradient between the main channel and the first outer channel of the device;
introducing and flowing a biological sample fluid into the other of the main channel or first side channel that is not flowing the chemo-modulatory fluid;
allowing and detecting migration of target cells and non-target cells in the biological sample fluid in response to the chemogradient, such that target cells in the sample move toward or away from the chemo-modulatory fluid in response to the chemogradient, non-target cells in the sample migrate toward or away from the chemo-modulatory fluid in response to the chemogradient, or both, and wherein the movement of the target cells is distinguishable from the movement of the non-target cells based on one or more of speed and distance of migration of the cells across the chemogradient in the device via the plurality of microchannels; and
introducing and flowing a flushing fluid from the first flushing port through the one or more first-side collection channels such that any target or non-target cells located in the one or more first-side collection channels between the main channel and the first outer channel are flushed by the flushing fluid to the individual collection outlets and are separated into different collection outlets based on distance of migration within the device.

15. The method of claim 14, further comprising subtyping any target or non-target cells collected in the individual collection outlets based on distance migrated toward or away from the chemo-modulatory fluid.

16. A method for enriching target cells in a biological sample, the method comprising:
in the device of claim 6, introducing a chemo-modulatory fluid in either the first inlet of the main channel or in both of the second and third inlets of the first and second outer channels, respectively, such that the chemo-modulatory fluid flows in the device and establishes a chemogradient between the main channel and the first and second outer channels of the device;
introducing and flowing a biological sample fluid comprising target cells into the other of the main or first and second outer channel that is not flowing the chemo-modulatory fluid;
allowing and detecting migration of target cells and non-target cells in the biological sample fluid in response to the chemogradient, such that target cells in the sample move toward or away from the chemo-modulatory fluid in response to the chemogradient, non-target cells in the sample migrate toward or away from the chemo-modulatory fluid in response to the chemogradient, or both, and wherein the movement of the target cells is distinguishable from the movement of the non-target cells based on one or more of speed and distance of migration of the cells across the chemogradient in the device via the plurality of microchannels; and
introducing and flowing a flushing fluid from either or both the first and second flushing port through either or both of the one or more first-side collection channels and one or more second-side collection channels such that any target or non-target cells located in the one or more first-side and second-side collection channels between the main channel and the first and second outer channels are flushed by the flushing fluid to the individual collection outlets and are separated into different collection outlets based on distance of migration within the device.

17. The method of claim 16, wherein
the chemo-modulatory fluid is the first fluid and is flowed from the first inlet through the main channel such that a chemogradient is established between the main channel and each of the first and second outer channels;
the biological sample fluid comprises a first and second biological sample fluid, wherein the first and second biological sample fluids are the same or different, the first biological sample fluid is flowed from the second inlet through the first outer channel and the second biological sample fluid is flowed from the third inlet through the second outer channel, such that target cells in each of the first and second sample fluids move toward or away from the main channel via the first and second plurality of micro channels in response to the chemogradient.

18. The method of claim 17, wherein the chemo-modulatory fluid comprises a chemo-attractant for the target cells and optionally comprises a chemo-repellent for non-target cells, such that the target cells move toward the chemo-modulatory fluid in the main channel in response to the chemogradient, and wherein the method further comprises sub-typing the target cells based on the speed and distance migrated from the first or second outer channel toward the main channel.

19. The method of claim 18, wherein the target cells are circulating tumor cells (CTCs) and wherein non-target cells are white blood cells (WBC's) and wherein the chemo-modulatory fluid comprises as chemo-attractant for CTC's and a chemo-repellent for WBC's.

20. The method of claim 16, wherein
the biological sample fluid is the first fluid and is flowed from the first inlet through the main channel;
the chemo-modulatory fluid comprises a first and second chemo-modulatory fluid, wherein the first and second chemo-modulatory fluids are the same or different,
the first chemo-modulatory fluid is flowed from the second inlet through the first outer channel such that a first chemogradient is established between the first outer channel and the main channel, and the second chemo-modulatory fluid is flowed from the third inlet through the second outer channel, such that a second chemogradient is established between the first outer channel and the main channel, and wherein target cells in the biological sample fluid move from the main channel toward or away from the first and second outer channels via the first and second plurality of micro channels in response to the first and second chemogradients.

21. The method of claim 20, wherein the first and second chemo-modulatory fluids are the same and comprise a chemo-attractant for the target cells and optionally comprise a chemo-repellent for non-target cells, such that the target cells move toward the first and second chemo-modulatory fluids in the first and second outer channels in response to the first and second chemogradients, and wherein the method further comprises sub-typing the target cells based on the speed and distance migrated toward the first or second outer channel.

22. The method of claim 20, wherein the first and second chemo-modulatory fluids are different, wherein the first chemo-modulatory fluid comprises a chemo-attractant for the target cells and optionally comprises a chemo-repellent for non-target cells, and wherein the second chemo-modulatory fluid comprises a chemo-attractant for non-target cells, such that the target cells move toward the first outer channel in response to the first chemogradient and the non-target cells remain in the main channel or move toward the second outer channel in response to the second chemogradient.

23. The method of claim 22, wherein the target cells are circulating tumor cells (CTCs) and wherein non-target cells are white blood cells (WBC's), wherein the first chemo-modulatory fluid comprises a chemo-attractant for CTC's and optionally comprises a chemo-repellent for WBC's, and wherein the second chemo-modulatory fluid comprises a chemo-attractant for WBC's.
